(19) United States
(12) Patent Application Publication
(10) Pub. No.: US 2015/0094474 A1
(43) Pub. Date: Apr. 2, 2015

(54) AGRICULTURAL CHEMICALS

(71) Applicant: Redx Pharma Ltd., Liverpool (GB)

(72) Inventors: William Thompson, Liverpool (GB); Peter Jackson, Liverpool (GB); Derek Lindsay, Liverpool (GB); Thomas Screen, Leeds (GB); Benjamin Moulton, Leeds (GB); Christopher Urch, Little Thetford (GB)

(21) Appl. No.: 14/383,777

(22) PCT Filed: Mar. 13, 2013
(86) PCT No.: PCT/GB2013/050621

§ 371 Date: Sep. 8, 2014

(30) Foreign Application Priority Data
Mar. 13, 2012 (GB) 1204382.4
Dec. 4, 2012 (GB) 1221788.1

(51) Int. Cl. A01N 53/00; C07C 255/44; C07C 255/61; A01N 37/34; C07C 255/40; A01N 43/56; A01N 35/02; C07D 211/32; A01N 47/16; C07D 211/34; C07D 401/04; C07C 69/743; C07C 49/255

(52) U.S. Cl. CPC A01N 53/00; C07C 69/743; C07C 255/44; C07C 255/61; A01N 37/34; C07C 255/40; C07C 49/255; A01N 35/02; C07D 211/32; A01N 47/16; C07D 211/34; C07D 401/04; A01N 43/56
USPC 546/245; 560/124; 558/407; 558/408; 558/410; 568/329; 546/275.4

(57) ABSTRACT

The present invention relates to derivatives of compounds which are known to be of use in the field of agriculture. These derivatives are differentiated from the parent active compound by virtue of being redox derivatives of the active compound. This means that one or more of the functional groups in the active compound has been converted to another group in one or more changes one or more of which may be considered to represent a change of oxidation state relative to the groups in the original compound. We refer to these compounds generally as redox derivatives. The compounds are of use as insecticides, herbicides and insect repellents.

The present invention relates to derivatives of compounds which are known to be of use in the field of agriculture. These derivatives are differentiated from the parent active compound by virtue of being redox derivatives of the active compound. This means that one or more of the functional groups in the active compound has been converted to another group in one or more changes one or more of which may be considered to represent a change of oxidation state relative to the groups in the original compound. We refer to these compounds generally as redox derivatives.

Given the global increase in demand for food, there is an international need for new treatments to reduce food crop losses to disease, insects and weeds. Over 40% of crops are lost before harvest, and 10% post harvest, worldwide. Losses have actually increased since the mid-1990s.

A new threat contributing to this is the emergence of chemical-resistant organisms, for example glyphosate-resistant weeds in USA.

Many current crop protection products are harmful and may cause acute and chronic health effects in those who are exposed, ranging from irritation of the skin and eyes to more severe effects such as disruption to the central nervous system and cancer. Strong evidence has also linked exposure to birth defects, foetal death, and abnormal neurodevelopment.

The WHO estimate that each year, 3 million agricultural workers in the developing world experience severe poisoning from crop protection chemicals, with 18,000 deaths. As many as 25 million workers in developing countries may suffer mild poisoning each year.

Crop protection chemicals are a major source of long-term environmental pollution. It is estimated that 98% of insecticides and 95% of herbicides impact species other than the direct target, and contaminate local air, water and soil. Many chemicals do not degrade and are persistent organic pollutants.

Excessive use can reduce biodiversity, reduce nitrogen fixation, contribute to pollinator decline, destroy nesting habitat for birds and threaten endangered species. Organisms can also develop a resistance to the chemical, requiring a greater dose of the pesticide to be used to counteract the resistance, cause a spiraling of the pollution problem.

An aim of the present invention is to provide pesticides (e.g. herbicides, insecticides and insect repellents) which have activity either non selectively, i.e. broad spectrum activity, or which are active specifically against selective target organisms.

An aim of the present invention is to provide compounds which are less persistant in the environment after use than the parent active.

Alternatively or additionally the compounds of the present invention are less prone to bioaccumulation once in the food chain than the parent active.

Another aim of the invention is to provide compounds which are less harmful to humans than the parent active.

Alternatively or additionally, the compounds of the invention may be less harmful than the parent active to one or more of the following groups: amphibians, fish, mammals (including domesticated animals such as dogs, cats, cows, sheep, pigs, goats, etc), reptiles, birds, and beneficial invertebrates (e.g. insects or worms), nematodes, beneficial fungi and nitrogen-fixing bacteria.

The compounds of the invention may be as active or more active than the parent active. They may have activity against organisms which have developed a resistance to the parent active. However, the present invention also concerns such redox derivatives of active compounds which have only a low level activity relative to that of the parent compound. These lower activity compounds are still effective as insecticides, insect repellents and/or herbicides but have other advantages relative to existing compounds such as, for example, a reduced environmental impact.

The compounds of the invention may be more selective than the parent, i.e. they may have better, similar or even slightly lower activity than the parent against target species but have a significantly lower activity against non-target species (e.g. the crops which are being protected).

The derivatives of the invention may be related to the original parent active agriculturally useful compound by only a single change, or may be related via several changes including one or more changes of oxidation state. In certain cases, the functional group obtained after two or more transformations may be in the same oxidation state as the parent active compound (and we include these compounds in our definition of redox derivatives). In other cases, the oxidation state of the derivative of the invention may be regarded as being different from that of the parent compound.

Generally, the present invention thus relates to redox derivatives which have the same type of activity i.e. against the same targets as the parent known active compound itself does. In some instances, the compounds may have new activity against a different target also in addition to that of the parent, or may have activity against a different target in preference to that of the parent. It is generally intended however that the activity of the compounds of the invention is the same in terms of its type as that of its respective ultimate parent compound i.e. the known active compound upon which the redox compound of the invention is ultimately based.

This invention provides compounds that achieve one or more of the above aims. The compounds may be active in their own right or may metabolise or react in aqueous media to yield a parent active compound. Ultimately, the overall skeleton i.e. gross structure of the parent active molecule is substantially retained but the various functional groups have been modified and we have identified “islands of activity” in these new genera of compounds. The activity of these compounds of the present invention cannot be predicted empirically based on knowledge of the respective parent compounds because the change of potency of an inhibitor depends on the binding of the inhibitor to the protein and it's ability to reach the protein.

### SUMMARY OF THE INVENTION

In a first aspect of the invention there is provided a compound of formula I:

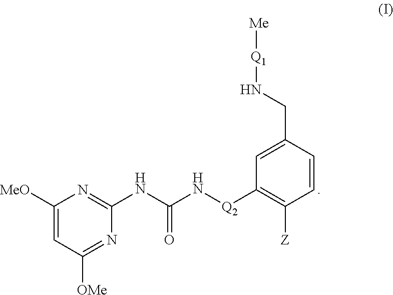

wherein Z is independently selected from the group CHO, CH═NOR³, CH(OR⁶)(OR⁶), heteroaryl, CH₂OR⁴;
Q₁ and Q₂ are independently selected from S(O) and S(O)₂;
R³ is independently a group selected from: H, C₁-C₄ alkyl, C₁-C₄ haloalkyl, phenyl, benzyl;
R⁴ is independently a group selected from H and Ac;
R⁶ is independently at each occurrence a group selected from C₁-C₄ alkyl, benzyl; or two R⁶ groups together with the atoms to which they are attached form a 5- or 6-membered ring;
wherein each of the aforementioned alkyl, haloalkyl, phenyl, benzyl and heteroaryl groups are optionally substituted, where chemically possible, by 1 to 3 substituents which are independently at each occurrence selected from: oxo, imino, oximo, halo, nitro, cyano, hydroxyl, amino, CO₂H, CO₂—(C₁-C₄alkyl), C(O)H, C₁-C₄-alkyl, C₁-C₄ haloalkyl, C₁-C₄ alkoxy, and C₁-C₄ haloalkoxy.

In an embodiment, Z is independently selected from CHO and CH═NOR³. In an embodiment, Z is CHO. In an alternative embodiment, Z is CH═NOR³. In this embodiment, R³ may be H. Alternatively, R³ may be C₁-C₄ alkyl, e.g. R³ may be methyl or ethyl.

In a particular embodiment, Z is CH₂OR⁴. Thus, R⁴ may be H. Alternatively, R⁴ may be Ac.

In an alternative embodiment, Z may be heteroaryl. Thus, Z may be a five membered heteroaryl group, i.e. Z may be pyrrole, furan, thiophene, pyrazole, imidazole, oxazole, isoxazole, triazole, oxadiazole, thiodiazole, tetrazole. In an embodiment, Z may be pyrrole, furan, thiophene, pyrazole, imidazole, oxazole, isoxazole, oxadiazole, thiodiazole.

In an embodiment, Q₁ is S(O)₂.

In an embodiment, Q₂ is S(O)₂.

In a particular embodiment, Q₁ and Q₂ are both S(O)₂.

In an embodiment, the compound of formula I is a compound selected from:

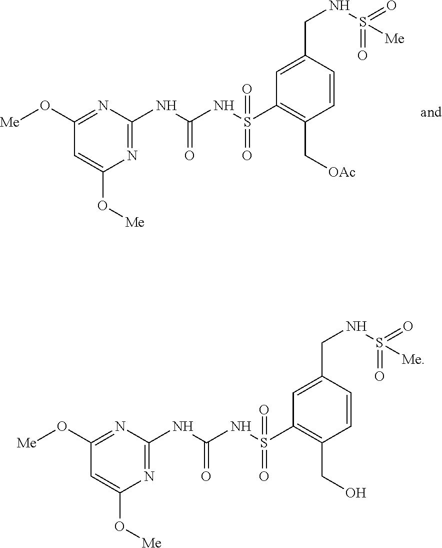 [SMILES: COc1cc(OC)nc(NC(=O)NS(=O)(=O)c2cc(CNS(C)(=O)=O)ccc2COC(C)=O)n1 ; COc1cc(OC)nc(NC(=O)NS(=O)(=O)c2cc(CNS(C)(=O)=O)ccc2CO)n1]

Compounds of the first aspect of the invention are based on mesosulfuron and may be used as herbicides. Mesosulfuron is an acetolactate synthase (ALS) inhibitor which blocks the synthesis of branched chain amine acids (leucine, valine, isoleucine). It is envisaged that the compounds of formula I will likewise be ALS inhibitors and herbicides or will under conditions of use convert to a compound having this sort of activity.

In a second aspect of the invention there is provided a compound of formula IIa:

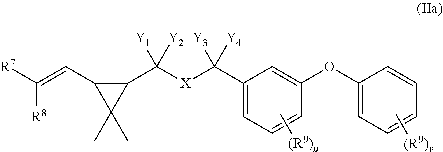

wherein X is NH, CH₂ or O;
wherein Y₁ is H and Y₂ is a group independently selected from W, OR⁵ and H and Y₃ and Y₄ together form a group independently selected from: ═O and ═NOR³; or
Y₃ is H and Y₄ is a group independently selected from W, OR⁵ and H and Y₁ and Y₂ together form a group independently selected from: ═O and ═NOR³; or
wherein

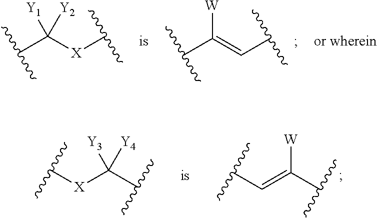

wherein W is a group independently selected from: H, CN, CO₂R⁵, CHO, CH═NOR³, CH(OR⁶)(OR⁶), CSNHR⁵, CH₂OR⁴, CONHR⁵;
or Y₂ and W, the atoms to which they are attached and the oxygen atom between the point of attachment of W and Y₂ together form a five membered ring in which two of the atoms in the ring are oxygen, and wherein the ring is optionally substituted with a group selected from: ═O or OR⁵;
R³ is independently a group selected from: H, C₁-C₄ alkyl, C₁-C₄ haloalkyl, phenyl, benzyl;
R⁴ is independently a group selected from: H and Ac;
R⁵ is independently at each occurrence a group selected from: H, C₁-C₄ alkyl, phenyl, benzyl;
R⁶ is independently at each occurrence a group selected from: C₁-C₄ alkyl, benzyl; or two R⁶ groups together with the atoms to which they are attached form a 5- or 6-membered ring;
R⁷ and R⁸ are a group independently selected from: halo and C₁-C₄ haloalkyl;
R⁹ is independently at each occurrence a group selected from: halo, C₁-C₄ alkyl, C₁-C₄-haloalkyl;
wherein each of the aforementioned alkyl, haloalkyl, phenyl and benzyl groups are optionally substituted, where chemically possible, by 1 to 3 substituents which are independently at each occurrence selected from: oxo, imino, oximo, halo, nitro, cyano, hydroxyl, amino, CO₂H, CO₂—(C₁-C₄alkyl), C(O)H, C₁-C₄-alkyl, C₁-C₄ haloalkyl, C₁-C₄ alkoxy, and C₁-C₄ haloalkoxy;
u is an integer selected from: 0, 1, 2, 3, 4; and
v is an integer selected from: 0, 1, 2, 3, 4, 5;
with the proviso that the compound is not a compound selected from:

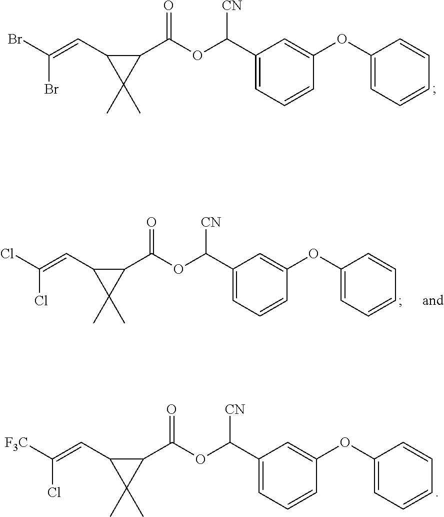 [SMILES: CC1(C)C(C=C(Br)Br)C1C(=O)OC(C#N)c1cccc(Oc2ccccc2)c1 ; CC1(C)C(C=C(Cl)Cl)C1C(=O)OC(C#N)c1cccc(Oc2ccccc2)c1 ; CC1(C)C(/C=C(\Cl)C(F)(F)F)C1C(=O)OC(C#N)c1cccc(Oc2ccccc2)c1]

In an embodiment, Y₁ is H and Y₂ is a group independently selected from W, OR⁵ and H and Y₃ and Y₄ together form a group independently selected from: ═O and ═NOR³; or Y₃ is H and Y₄ is a group independently selected from W, OR⁵ and H and Y₁ and Y₂ together form a group independently selected from: ═O and ═NOR³.

In an embodiment, the compound of formula IIa is a compound of formula IIb:

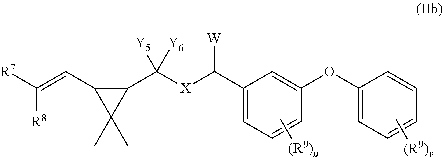

wherein X is O or NH;
Y₅ is H and Y₆ is a group independently selected from OR⁵ and H;
or Y₅ and Y₆ together form a group independently selected from: ═O and ═NOR³;
wherein W is a group independently selected from: H, CN, CO₂R⁵, CHO, CH═NOR³, CH(OR⁶)(OR⁶), CH₂OR⁴, CONHR⁵;
or Y₆ and W, the atoms to which they are attached and the oxygen atom between the point of attachment of W and Y₆ together form a five membered ring in which two of the atoms in the ring are oxygen, and wherein the ring is optionally substituted with a group selected from: ═O or OR⁵;
R³ is independently a group selected from: H, C₁-C₄ alkyl, C₁-C₄ haloalkyl, phenyl, benzyl;
R⁴ is independently a group selected from: H and Ac;
R⁵ is independently at each occurrence a group selected from: H, C₁-C₄ alkyl, phenyl, benzyl;
R⁶ is independently at each occurrence a group selected from: C₁-C₄ alkyl, benzyl; or two R⁶ groups together with the atoms to which they are attached form a 5- or 6-membered ring;
R⁷ and R⁸ are a group independently selected from: halo and C₁-C₄ haloalkyl;
R⁹ is independently at each occurrence a group selected from: halo, C₁-C₄ alkyl, C₁-C₄-haloalkyl;
wherein each of the aforementioned alkyl, haloalkyl, phenyl and benzyl groups are optionally substituted, where chemically possible, by 1 to 3 substituents which are independently at each occurrence selected from: oxo, imino, oximo, halo, nitro, cyano, hydroxyl, amino, CO₂H, CO₂—(C₁-C₄alkyl), C(O)H, C₁-C₄-alkyl, C₁-C₄ haloalkyl, C₁-C₄ alkoxy, and C₁-C₄ haloalkoxy;
u is an integer selected from: 0, 1, 2, 3, 4; and
v is an integer selected from: 0, 1, 2, 3, 4, 5;
with the proviso that the compound is not a compound selected from:

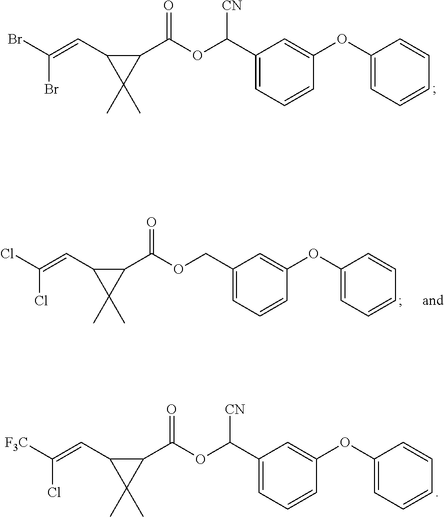 [SMILES: CC1(C)C(C=C(Br)Br)C1C(=O)OC(C#N)c1cccc(Oc2ccccc2)c1 ; CC1(C)C(C=C(Cl)Cl)C1C(=O)OCc1cccc(Oc2ccccc2)c1 ; CC1(C)C(/C=C(\Cl)C(F)(F)F)C1C(=O)OC(C#N)c1cccc(Oc2ccccc2)c1]

In an embodiment, the compound of formula IIa is a compound of formula IIc:

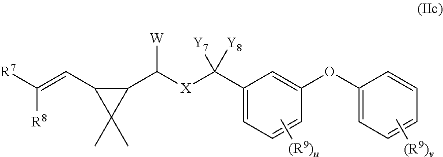

wherein Y₇ is H and Y₈ is a group independently selected from OR⁵ and H;
or Y₇ and Y₈ together form a group independently selected from: ═O and ═NOR³;
wherein W is a group independently selected from: H, CN, CO₂R⁵, CHO, CH═NOR³, CH(OR⁶)(OR⁶), CH₂OR⁴, CONHR⁵;
or Y₈ and W, the atoms to which they are attached and the oxygen atom between the point of attachment of W and Y₈ together form a five membered ring in which two of the atoms in the ring are oxygen, and wherein the ring is optionally substituted with a group selected from: ═O or OR⁵;
R³ is independently at each occurrence a group selected from: H, C₁-C₄ alkyl, C₁-C₄ haloalkyl, phenyl, benzyl;
R⁴ is independently at each occurrence a group selected from: H and Ac;
R⁵ is independently at each occurrence a group selected from: H, C₁-C₄ alkyl, phenyl, benzyl;
R⁶ is independently at each occurrence a group selected from: C₁-C₄ alkyl, benzyl; or two R⁶ groups together with the atoms to which they are attached form a 5- or 6-membered ring;
R⁷ and R⁸ are a group independently selected from: halo and C₁-C₄ haloalkyl;
R⁹ is independently at each occurrence a group selected from: halo, C₁-C₄ alkyl, C₁-C₄-haloalkyl;
wherein each of the aforementioned alkyl, haloalkyl, phenyl and benzyl groups are optionally substituted, where chemically possible, by 1 to 3 substituents which are independently at each occurrence selected from: oxo, imino, oximo, halo, nitro, cyano, hydroxyl, amino, CO₂H, CO₂—(C₁-C₄alkyl), C(O)H, C₁-C₄-alkyl, C₁-C₄ haloalkyl, C₁-C₄ alkoxy, and C₁-C₄ haloalkoxy;
u is an integer selected from: 0, 1, 2, 3, 4; and
v is an integer selected from: 0, 1, 2, 3, 4, 5.

In an embodiment, the compound of formula IIa is a compound of formula IId:

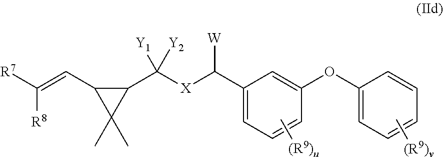

wherein R⁷, R⁸, Y₁, Y₂, X, W, R⁹, u and v are as described above for formula IIa.

In an embodiment, applicable to compounds of formulae IIa, IIb and IIc u is 1. Preferably, u is 0. In an embodiment v is 1. Preferably v is 0.

In an embodiment, the compound of formula IIa is a compound of formula IIIa:

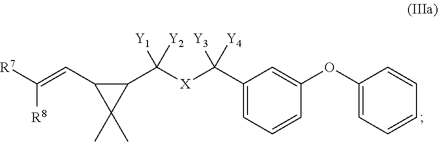

wherein R⁷, R⁸, X, Y₁, Y₂, Y₃ and Y₄ are as described above.

In an embodiment, the compound of formula IIa is a compound of formula IIIb:

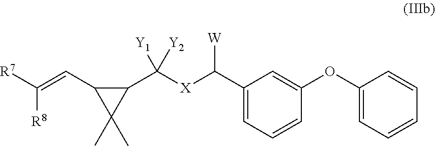

wherein R⁷, R⁸, X, Y₅, Y₆ and W are as described above.

In an embodiment, the compound of formula IIa is a compound of formula IIIc:

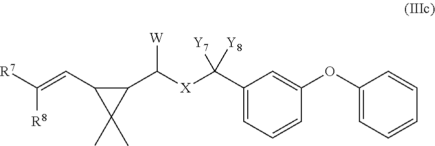

wherein R⁷, R⁸, X, Y₇, Y₈ and W are as described above.

In an embodiment, applicable to compounds of formulae IIa-IIIc, R⁷ is halo. Thus, R⁷ may be Br. Alternatively, R⁷ may be Cl. Alternatively, R⁷ may be F. In an alternative embodiment, applicable to compounds of formulae IIa-IIIc, R⁷ is C₁-C₄-haloalkyl (e.g. C₁-C₄ fluoroalkyl). In a particular embodiment, R⁷ is CF₃.

In an embodiment, applicable to compounds of formulae IIa-IIIc, R⁸ is halo. Thus, R⁸ may be Br. Alternatively, R⁸ may be Cl. Alternatively, R⁸ may be F. In an alternative embodiment, applicable to compounds of formulae IIa-IIIc, R⁸ is C₁-C₄-haloalkyl (e.g. C₁-C₄ fluoroalkyl). In a particular embodiment, R⁸ is CF₃.

In an embodiment, the compound of formula IIa is a compound of formulae IVa, Va or VIa:

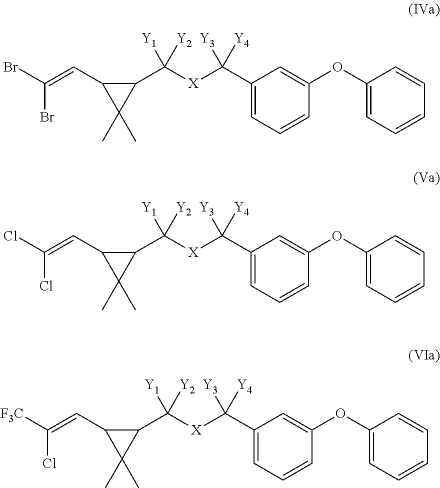

wherein X, Y₁, Y₂, Y₃ and Y₄ are as described above.

In an embodiment, the compound of formula IIa is a compound of formulae IVb, Vb or VIb:

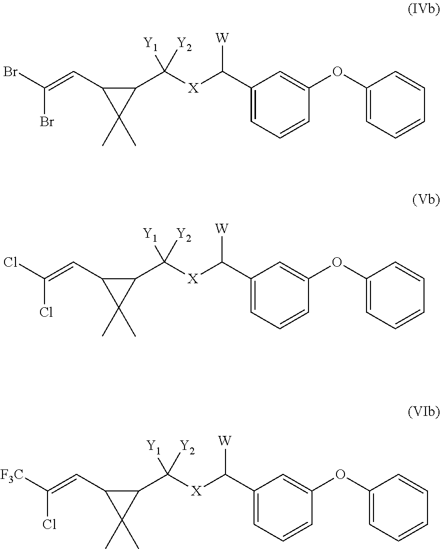

wherein X, Y₁, Y₂, and W are as described above.

In an embodiment, the compound of formula IIa is a compound of formulae IVc, Vc or VIc:

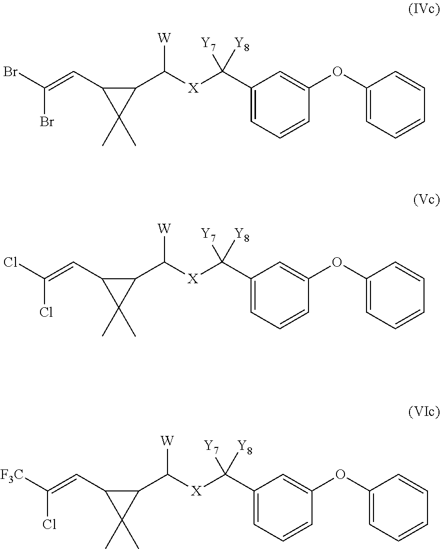

wherein X, Y₇, Y₈, and W are as described above.

In an embodiment, the compound of formula IIa is a compound of formula IVa. In an embodiment, the compound of formula IIa is a compound of formula Va. In an embodiment, the compound of formula IIa is a compound of formula VIa.

In an embodiment, the compound of formula IIa is a compound of formula IVb. In an embodiment, the compound of formula IIa is a compound of formula Vb. In an embodiment, the compound of formula IIa is a compound of formula VIb.

In an embodiment, the compound of formula IIa is a compound of formula IVc. In an embodiment, the compound of formula IIa is a compound of formula Vc. In an embodiment, the compound of formula IIa is a compound of formula VIc.

In an embodiment, applicable to any of formula IIa-VIc, X is O. In an alternative embodiment, X is NH. In a further alternative, X is CH₂. In another embodiment, X is selected from NH or CH₂.

Thus, for example, for compounds of formula IIb, it may be that X is O.

In an embodiment, applicable to any of formulae IIa-VIc, W is CN. In an alternative embodiment, W may be H. In an embodiment, W is not H. In a further embodiment W is CO₂R⁵. R⁵ may be H or R⁵ may be C₁-C₄ alkyl, e.g. ethyl.

In an embodiment, applicable to formula IIb, Y₅ and Y₆ together form ═O. In a further embodiment, Y₅ and Y₆ together form ═O and X is NH.

In an embodiment, applicable to any of formulae IIa, IId, IIIa, IIIb, IVa, IVb, Va, Vb, VIa and VIb, Y₁ and Y₂ together form ═O. In a further embodiment, Y₁ and Y₂ together form ═O and X is NH.

In an embodiment, applicable to any of formulae IIc, IIIc, IVc, Vc and VIc, Y₇ and Y₈ together form ═O.

In an embodiment, applicable to formula IIb, the group

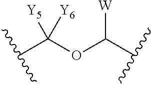

is selected from:

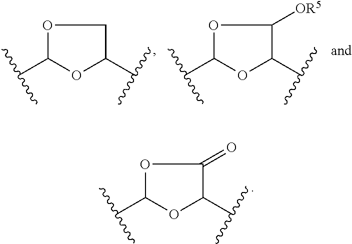

In an embodiment, the compound of formula IIa is a compound of formula VII, VIII or IX

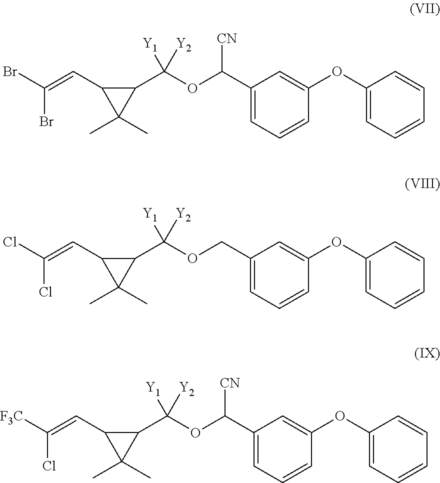

wherein Y₁ and Y₂ are as described above.

In an embodiment, the compound of formula IIa is a compound of formula VII. In an embodiment, the compound of formula IIa is a compound of formula VIII. In an embodiment, the compound of formula IIa is a compound of formula IX.

In an embodiment, the compound of formula IIa is a compound of formula VIIa, VIIIa or IXa

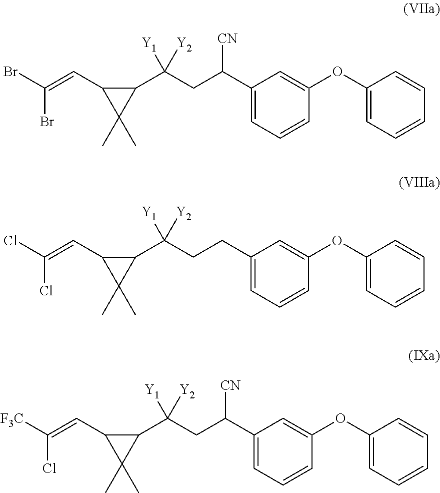

wherein Y₁ and Y₂ are as described above. In an embodiment, the compound of formula IIa is a compound of formula VIIa. In an embodiment, the compound of formula IIa is a compound of formula VIIIa. In an embodiment, the compound of formula IIa is a compound of formula IXa. In an embodiment, applicable to any of formulae VIIa, VIIIa and IXa, Y₁ and Y₂ together form ═O.

In an embodiment, the compound of formula IIa is a compound of formula VIIb, VIIIb or IXb

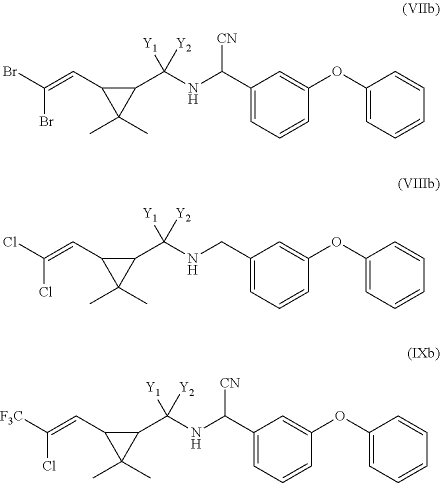

wherein Y₁ and Y₂ are as described above. In an embodiment, the compound of formula IIa is a compound of formula VIIb. In an embodiment, the compound of formula IIa is a compound of formula VIIIb. In an embodiment, the compound of formula IIa is a compound of formula IXb. In an embodiment, applicable to any of formulae VIIb, VIIIb and IXb, Y₁ and Y₂ together form ═O.

In an embodiment, applicable to any of formulae IIa, IId, IIIa, IIIb, IVa, IVb, Va, Vb, VIa and VIb, X is CH₂ and Y₁ and Y₂ together form ═O. Thus, it may be that

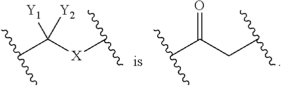

Additionally, where Y₁ and Y₂ together form ═O,

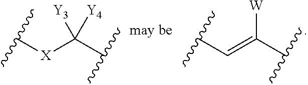

In a further embodiment, W is H.

In an embodiment, applicable to any of formulae IIb X is CH₂ and Y₅ and Y₆ together form ═O. Thus, it may be that

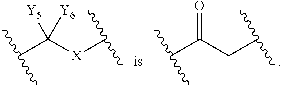

In an embodiment, applicable to any of formulae IIa, IId, IIIa, IIIb, IVa, IVb, Va, Vb, VIa, VIb, VII, VIIa, VIIb, VIII, VIIIa, VIIIb, IX, IXa and IXb, Y₁ is H. In yet another alternative embodiment, Y₁ is OR⁵. In this embodiment, R⁵ may be H. Alternatively, R⁵ may not be H. R⁵ may be C₁-C₄ alkyl. Thus, R⁵ may be ethyl or R⁵ may be methyl. In yet another embodiment, Y₁ and Y₂ together form ═O.

In an embodiment, applicable to formula IIb, Y₆ is H. In yet another alternative embodiment, Y₆ is OR⁵. In this embodiment, R⁵ may be H. Alternatively, R⁵ may not be H. R⁵ may be C₁-C₄ alkyl. Thus, R₅ may be ethyl or R₅ may be methyl. In yet another embodiment, Y₁ and Y₂ together form ═O.

In an embodiment, applicable to any of formulae IIa, IId, IIIa, IIIb, IVa, IVb, Va, Vb, VIa, VIb, where X is O, Y₁ and Y₂ together do not form ═O. In an embodiment, applicable to VII, VIII and IX, Y₁ and Y₂ together do not form ═O.

In an embodiment, applicable to formula IIb where X is O, Y₅ and Y₆ together do not form ═O.

In an embodiment, the compound of formula IIa is a compound selected from:

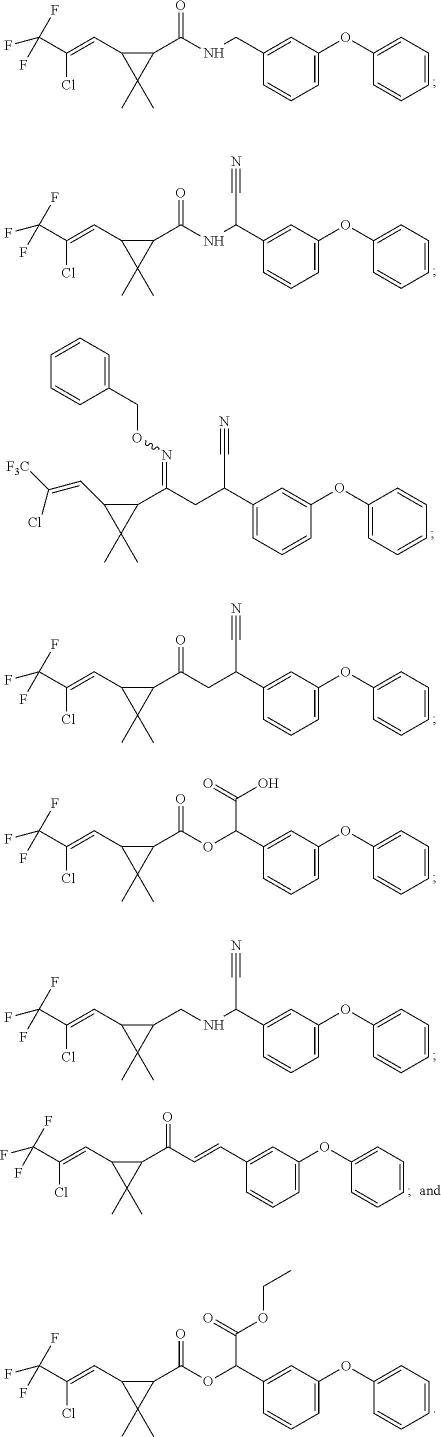

The compounds of the second aspect of the invention are based on permethrin, deltamethrin and cyhalothrin. They may be used as insecticides. They may be used to treat tick infestations in an animal or animal population. They may also be used to kill or repel mosquitoes, for instance in the prevention of diseases such as malaria, dengue fever and/or West Nile virus. They may be used in pest control. For instance they may be used in the control of pests such as ants, cockroaches, bedbugs, carpenter bees, spider mites, caterpillars, aphids, beetles. It is envisaged that the compounds of formulae II-IX will likewise have insecticidal activity or will under conditions of use convert to a compound having this sort of activity. We have demonstrated that compounds of this aspect have activity against aphids, cabbage moth caterpillars, spider mites and mosquito larvae.

In a third aspect of the invention is provided a compound of formula X:

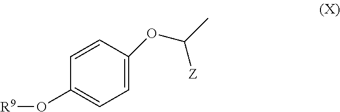

wherein Z is a group independently selected from: CHO, CH═NOR³, CH(OR⁶)(OR⁶), CH₂OR⁴;
R³ is independently a group selected from: H, C₁-C₄ alkyl, C₁-C₄ haloalkyl, phenyl, benzyl;
R⁴ is independently a group selected from: H and Ac;
R⁶ is independently at each occurrence a group selected from: C₁-C₄ alkyl, benzyl; or two R⁶ groups together with the atoms to which they are attached form a 5- or 6-membered ring;
R⁹ is a heteroaryl group;
wherein each of the aforementioned alkyl, haloalkyl, phenyl, benzyl and heteroaryl groups are optionally substituted, where chemically possible, by 1 to 3 substituents which are independently at each occurrence selected from: oxo, imino, oximo, halo, nitro, cyano, hydroxyl, amino, CO₂H, CO₂—(C₁-C₄alkyl), C(O)H, C₁-C₄-alkyl, C₁-C₄ haloalkyl, C₁-C₄ alkoxy, and C₁-C₄ haloalkoxy.

In an embodiment, the compound of formula X is a compound of formula XI or formula XII:

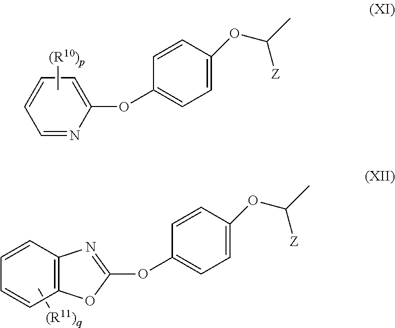

wherein Z is as described above;
R¹⁰ and R¹¹ are independently at each occurrence a group selected from: halo, C₁-C₄ alkyl, C₁-C₄-haloalkyl;
wherein each of the aforementioned alkyl and haloalkyl groups are optionally substituted,
where chemically possible, by 1 to 3 substituents which are independently at each occurrence selected from: oxo, imino, oximo, halo, nitro, cyano, hydroxyl, amino, CO₂H, CO₂—(C₁-C₄alkyl), C(O)H, C₁-C₄ haloalkyl, C₁-C₄ alkoxy, and C₁-C₄ haloalkoxy
p is an integer independently selected from: 0, 1, 2, 3, 4; and
q is an integer independently selected from: 0, 1, 2, 3, 4.

In an embodiment, the compound of formula X is a compound of formula XI. Alternatively, the compound of formula X is a compound of formula XII.

In an embodiment, R¹⁰ is halo. Thus, R¹⁰ may be Cl. Alternatively, R¹⁰ may be F. In an alternative embodiment, R¹⁰ is C₁-C₄-haloalkyl (e.g. C₁-C₄ fluoroalkyl). In a particular embodiment, R¹⁰ is CF₃.

In an embodiment, p is 0. Alternatively, p is an integer selected from: 1, 2, 3, 4. In a preferred embodiment p is 1. In an alternative preferred embodiment, p is 2.

In an embodiment, R¹¹ is halo. Thus, R¹¹ may be Cl. Alternatively, R¹¹ may be F. In an alternative embodiment, R¹¹ is C₁-C₄-haloalkyl (e.g. C₁-C₄ fluoroalkyl). Thus, R¹¹ may be CF₃.

In an embodiment, q is 0. Alternatively, q is an integer selected from: 1, 2, 3, 4. In a preferred embodiment q is 1.

In an embodiment, the compound of formula X is a compound of formulae XIII, XIV or XV:

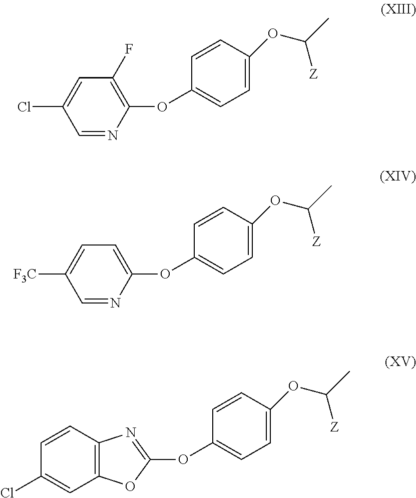

wherein Z is as described above.

In an embodiment, the compound of formula X is a compound of formula XIII. In another embodiment, the compound of formula X is a compound of formula XIV. In yet another embodiment, the compound of formula X is a compound of formula XV.

In an embodiment, applicable to compounds of any of formulae X-XV, Z is independently selected from CHO and CH═NOR³. In an embodiment, Z is CHO. In an alternative embodiment, Z is CH═NOR³. In this embodiment, R³ may be H. Alternatively, R³ may be C₁-C₄ alkyl, e.g. R³ may be methyl or R³ may be ethyl. In yet another alternative, R³ may be benzyl. Z may also be CH₂OR⁴. R⁴ may be H or R⁴ may be Ac.

In an embodiment, the compound of formula X is a compound selected from:

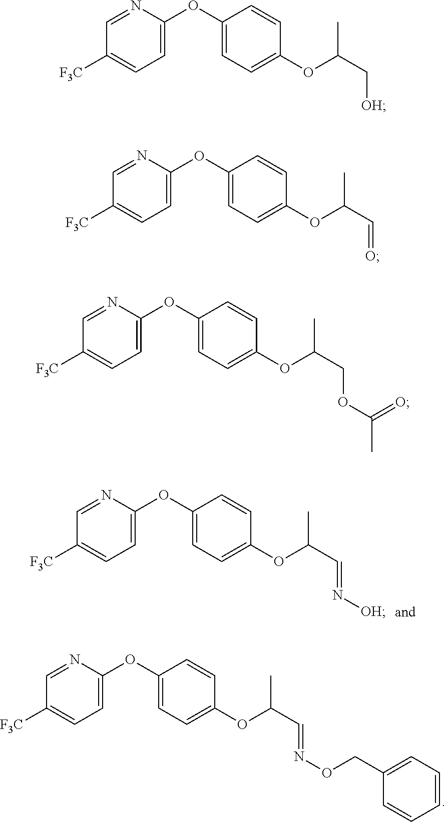 [SMILES: CC(CO)Oc1ccc(Oc2ccc(C(F)(F)F)cn2)cc1 ; CC(C=O)Oc1ccc(Oc2ccc(C(F)(F)F)cn2)cc1 ; CC(=O)OCC(C)Oc1ccc(Oc2ccc(C(F)(F)F)cn2)cc1 ; CC(/C=N/O)Oc1ccc(Oc2ccc(C(F)(F)F)cn2)cc1 ; CC(/C=N/OCc1ccccc1)Oc1ccc(Oc2ccc(C(F)(F)F)cn2)cc1]

The compounds of the third aspect of the invention are based on fenoxaprop, fluazifop and clodinafop. The compounds may be used as herbicides. Fenoxaprop, fluazifop and clodinafop inhibit acetyl CoA carboxylase and hence the biosynthesis of lipids. The active compounds contain carboxylic acids and are typically sold as esters. It is envisaged that the compounds of formulae X-XV will likewise inhibit acetyl CoA carboxylase and act as herbicides or will under conditions of use convert to a compound having this sort of activity.

In a fourth aspect of the invention is provided a compound of formula XVI:

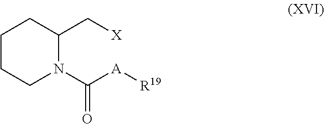

wherein X is a group independently selected from: CHO, CH═NOR³, CH(OR⁶)(OR⁶), CO₂R⁵;
A is a group selected from O, S and NH;
R³ is independently at each occurrence a group selected from: H, C₁-C₄ alkyl, C₁-C₄ haloalkyl, phenyl, benzyl;
R⁵ is independently at each occurrence a group selected from: H, C₁-C₄ alkyl, phenyl, benzyl;
R⁶ is independently at each occurrence a group selected from: C₁-C₄ alkyl, benzyl; or two R⁶ groups together with the atoms to which they are attached form a 5- or 6-membered ring; and
R¹⁹ is independently at each occurrence a group selected from: H, C₁-C₆ alkyl, C₁-C₄ haloalkyl, phenyl, benzyl;
wherein each of the aforementioned alkyl, haloalkyl, phenyl and benzyl groups are optionally substituted, where chemically possible, by 1 to 3 substituents which are independently at each occurrence selected from: oxo, imino, oximo, halo, nitro, cyano, hydroxyl, amino, CO₂H, CO₂—(C₁-C₄alkyl), C(O)H, C₁-C₄-alkyl, C₁-C₄ haloalkyl, C₁-C₄ alkoxy, and C₁-C₄ haloalkoxy.

In an embodiment, A is NH. In an alternative embodiment, A is O.

In an embodiment, R¹⁹ is C₁-C₆ alkyl. In a further embodiment, R¹⁹ is C₁-C₄ alkyl. Thus, R¹⁹ can be methyl, ethyl, isopropyl or n-propyl. In a particular embodiment, R¹⁹ is C₄ alkyl. In an embodiment, R¹⁹ is n-butyl or sec-butyl. Preferably, R¹⁹ is sec-butyl.

In an embodiment, the compound of formula XVI is a compound of formula XVII:

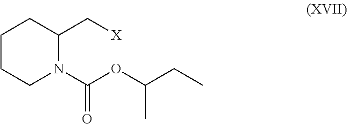

wherein X is as described above.

In an embodiment, applicable to compounds of formula XVI and XVII, X is a group independently selected from: CHO and CH═NOR³. In an embodiment, X is CHO. In an alternative embodiment, X is CH═NOR³. In this embodiment, R³ may be H. Alternatively, R³ may be C₁-C₄ alkyl, e.g. R³ may be methyl or R³ may be ethyl.

In an alternative embodiment, X is CO₂R⁵. Thus, R⁵ may be H. R⁵ may also be C1-C4-alkyl, e.g. methyl.

In an embodiment, the compound of formula XVI is a compound selected from:

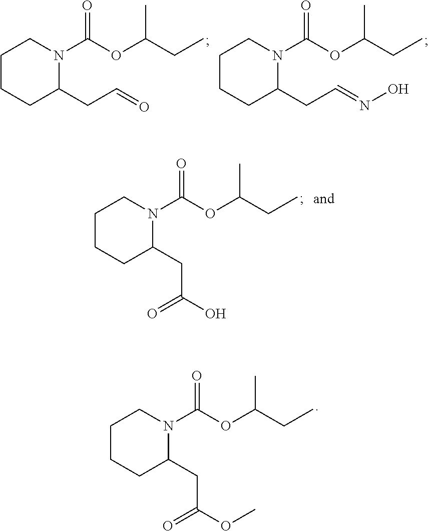

The compounds of the fourth aspect of the invention are based on icaridin. They may be used as insecticides or as an insect repellent. They may be used to repel mosquitoes, for instance in the prevention of diseases such as malaria, dengue fever and/or West Nile virus. They can also be used to repel ants, flies, cockroaches, aphids, spider mites, caterpillars. It is envisaged that the compounds of formula XVI and XVII will likewise be active as insecticides or likewise as an insect repellent as the parent active or will under conditions of use convert to a compound having this sort of activity. We have demonstrated that compounds of this aspect have activity against houseflies, cockroaches, ants and bedbugs.

In an fifth aspect of the invention is provided a compound of formula XVIII:

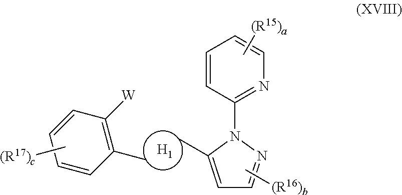

wherein

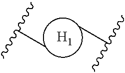

is a group selected from

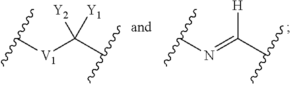

V₁ is a group independently selected from: O and NH;
Y₁ is H and Y₂ is independently at each occurrence a group selected from OR⁵ and H;
or Y₁ and Y₂ together form a group independently selected from: ═O and ═NOR³;
W is a group independently selected from: C(O)NR¹⁸R¹⁹, CHO, CO₂R⁵, CH═NOR³, CH(OR⁶)(OR⁶), heteroaryl, or CH₂OR⁴;
R³ is independently at each occurrence a group selected from: H, C₁-C₄ alkyl, C₁-C₄ haloalkyl, phenyl, benzyl;
R⁴ is independently a group selected from: H and Ac; R⁵ is independently at each occurrence a group selected from: H, C₁-C₄ alkyl, phenyl, benzyl;
R⁶ is independently at each occurrence a group selected from C₁-C₄ alkyl, benzyl; or two R⁶ groups together with the atoms to which they are attached form a 5- or 6-membered ring;
R¹⁵, R¹⁶ and R¹⁷ are independently at each occurrence a group selected from: halo, C₁-C₄ alkyl, C₁-C₄-haloalkyl and cyano;
R¹⁸ and R¹⁹ are independently at each occurrence a group selected from: H, C₁-C₄ alkyl, phenyl, benzyl;
wherein each of the aforementioned alkyl, haloalkyl, phenyl and benzyl groups are optionally substituted, where chemically possible, by 1 to 3 substituents which are independently at each occurrence selected from: oxo, imino, oximo, halo, nitro, cyano, hydroxyl, amino, CO₂H, CO₂—(C₁-C₄alkyl), C(O)H, C₁-C₄-alkyl, C₁-C₄ haloalkyl, C₁-C₄ alkoxy, and C₁-C₄ haloalkoxy;
a is an integer independently selected from: 0, 1, 2, 3, 4;
b is an integer independently selected from: 0, 1, 2;
c is an integer independently selected from: 0, 1, 2, 3, 4,
with the proviso that if Y₁ and Y₂ together form ═O and V₁ is NH, W is not C(O)NHMe.

In an embodiment, a is 0. In an alternative embodiment, a is independently selected from: 1, 2, 3, 4. Preferably, a is 1.

In an embodiment, b is 0. In an alternative embodiment, b is independently selected from: 1, 2. Preferably, b is 1.

In an embodiment, c is 0. In an alternative embodiment, c is independently selected from: 1, 2, 3, 4. Thus, c may be 1. Preferably, c is 2.

In an embodiment, the compound of formula XVIII is a compound of formula XIX:

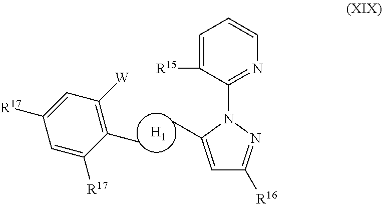

wherein

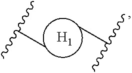

W, R¹⁵, R¹⁶ and R¹⁷ are as described above.

In an embodiment, applicable to compounds of formulae XVIII and XIX, R¹⁵ is independently at each occurrence selected from halo and C₁-C₄-haloalkyl. In an embodiment, R¹⁵ is independently at each occurrence halo. Thus, R¹⁵ may be Br and/or R¹⁵ may be Cl and/or R¹⁵ may be F. Preferably, R¹⁵ is Cl.

In an embodiment, applicable to compounds of formulae XVIII and XIX, R¹⁶ is independently at each occurrence halo. Thus, R¹⁶ may be Br or R¹⁶ may be Cl or R¹⁶ may be F. Preferably, R¹⁶ is Br.

In an embodiment, applicable to compounds of formulae XVIII and XIX, R¹⁷ is independently at each occurrence C₁-C₄ alkyl. Thus, R¹⁷ may be methyl or ethyl. Preferably, R¹⁷ is in at least one occurrence methyl. Alternatively or additionally, R¹⁷ is in at least one occurrence cyano.

In an embodiment, applicable to compounds of formulae XVIII and XIX, R¹⁸ is independently selected from C₁-C₄ alkyl and phenyl, benzyl. In a further embodiment, R¹⁸ is C₁-C₄ alkyl. Thus, R¹⁸ may be methyl or ethyl.

In an embodiment, the compound of formula XVIII is a compound of formula XX:

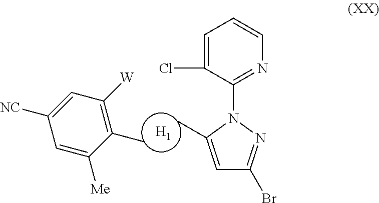

wherein

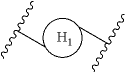

and W are as described above.

In an embodiment, the compound of formula XVIII is a compound of formula XXI or formula XXII:

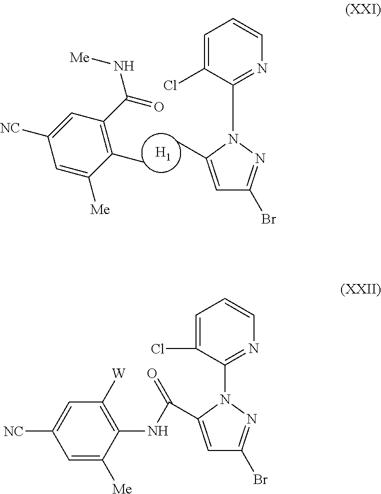

wherein

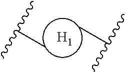

and W are as described above.

In an embodiment, the compound of formula XVIII is a compound of formula XXI. In an alternative embodiment, the compound of formula XVIII is a compound of formula XXII.

In an embodiment, applicable to compounds of formulae XVIII, XIX, XX and XXI,

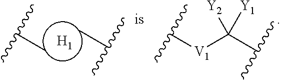

In an embodiment, applicable to compounds of formulae XVIII, XIX, XX and XXI,

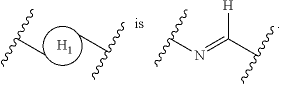

In an embodiment, applicable to compounds of formulae XVIII, XIX, XX and XXI, V₁ is NH.

In an embodiment, applicable to compounds of formulae XVIII, XIX, XX and XXI, Y₁ and Y₂ together form ═O. In an alternative embodiment, Y₂ is H. In a further alternative, Y₁ and Y₂ together form ═NOR³.

In yet another alternative embodiment, applicable to compounds of formulae XVIII, XIX, XX and XXI, Y₂ is OR⁵. In an embodiment, Y₂ is OR⁵ and V₁ is O. In an alternative embodiment, Y₂ is OR⁵ and V₁ is NH. In these embodiments, it may be that R⁵ is not H.

In an embodiment, applicable to compounds of formulae XVIII, XIX, XX and XXI, W is independently selected from CHO and CH═NOR³. In an embodiment, W is CHO. In an alternative embodiment, W is CH═NOR³. In this embodiment, R³ may be H. Alternatively, R³ may be C₁-C₄ alkyl, e.g. R³ may be methyl or R³ may be ethyl.

In an embodiment, applicable to compounds of formulae XVIII, XIX, XX and XXII, W is C(O)NR¹⁸R¹⁹. In a further embodiment, R¹⁹ is H. In another further embodiment, R¹⁸ is independently selected from C₁-C₄ alkyl and phenyl, benzyl. In yet another embodiment, R¹⁸ is C₁-C₄ alkyl. Thus, R¹⁸ may be methyl or ethyl, e.g. R¹⁸ may be methyl.

In an embodiment, applicable to compounds of formulae XVIII, XIX, XX and XXII, W is CO₂R⁵. Thus, R⁵ may be H. Alternatively R⁵ may be C₁-C₄ alkyl.

In an embodiment, applicable to compounds of formulae XVIII, XIX, XX and XXII, if Y₁ and Y₂ together form ═O, W is not C(O)NR¹⁸R¹⁹. In a further embodiment, if Y₁ and Y₂ together form ═O, W is neither C(O)NR¹⁸R¹⁹ nor CO₂H.

In an embodiment, the compound of formula XVIII is a compound selected from:

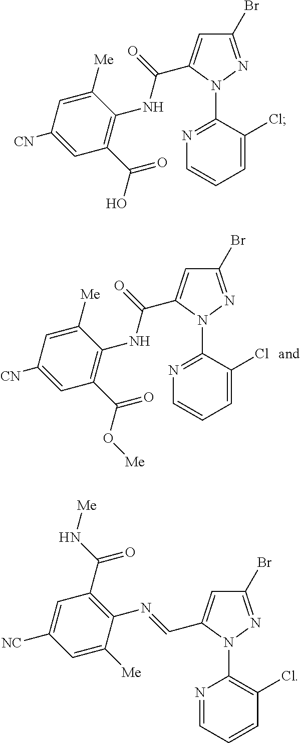 [SMILES: Cc1cc(C#N)cc(C(=O)O)c1NC(=O)c1cc(Br)nn1-c1ncccc1Cl ; COC(=O)c1cc(C#N)cc(C)c1NC(=O)c1cc(Br)nn1-c1ncccc1Cl ; CNC(=O)c1cc(C#N)cc(C)c1/N=C/c1cc(Br)nn1-c1ncccc1Cl]

In an alternative expression of the fifth aspect is provided a compound of formula XXIII:

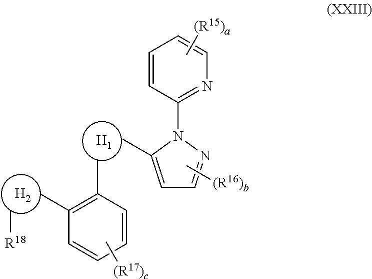

wherein

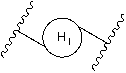

is a group selected from

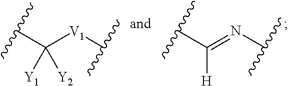

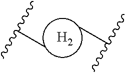

is a group selected from

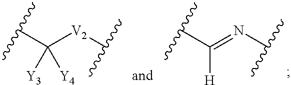

V₁ and V₂ are groups independently selected from: O and NH;
Y₁ and Y₃ are H and Y₂ and Y₄ are independently at each occurrence a group selected from OR⁵ and H;
or Y₁ and Y₂ together form a group independently selected from: ═O and ═NOR³; and/or
Y₃ and Y₄ together form a group independently selected from: ═O and ═NOR³;
R³ is independently at each occurrence a group selected from: H, C₁-C₄ alkyl, C₁-C₄ haloalkyl, phenyl, benzyl;
R⁵ is independently at each occurrence a group selected from: H, C₁-C₄ alkyl, phenyl, benzyl;
R¹⁵, R¹⁶ and R¹⁷ are independently at each occurrence a group selected from: halo, C₁-C₄ alkyl, C₁-C₄-haloalkyl and cyano;
R¹⁸ is a group independently selected from: H, C₁-C₄ alkyl, phenyl, benzyl;
wherein each of the aforementioned alkyl, haloalkyl, phenyl and benzyl groups are optionally substituted, where chemically possible, by 1 to 3 substituents which are independently at each occurrence selected from: oxo, imino, oximo, halo, nitro, cyano, hydroxyl, amino, CO₂H, CO₂—(C₁-C₄alkyl), C(O)H, C₁-C₄-alkyl, C₁-C₄ haloalkyl, C₁-C₄ alkoxy, and C₁-C₄ haloalkoxy;
a is an integer independently selected from: 0, 1, 2, 3, 4;
b is an integer independently selected from: 0, 1, 2;
c is an integer independently selected from: 0, 1, 2, 3, 4, with the proviso that if Y₁ and Y₂ together form ═O, Y₃ and Y₄ together form ═O and V₁ is NH then V₂ is not NH.

An alternative way of depicting formula (XXIII) is:

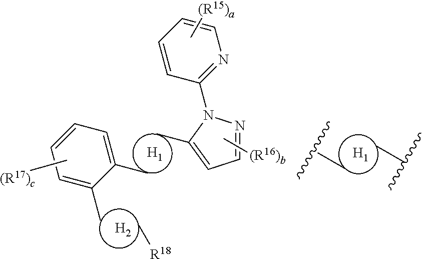

is a group selected from

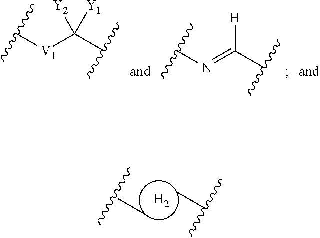

is a group selected from

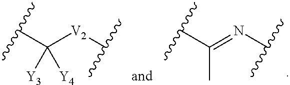

Obviously, in this alternative depiction, the definitions of R¹⁷, R¹⁸, R¹⁶, R¹⁵, a, b and c are the same as described above.

The compounds of the fifth aspect of the invention are based on cyantraniliprole, a ryanodine receptor agonist. They may be used as insecticides. It is envisaged that the compounds of formulae XVIII-XXIII will likewise be ryanodine receptor agonists and insecticides or will under conditions of use convert to a compound having this sort of activity. We have demonstrated that compounds of this aspect have activity against aphids, cabbage moth caterpillars, spider mites and mosquito larvae.

In any of the above aspects, heteroaryl groups may be independently selected from: 5 membered heteroaryl groups in which the heteroaromatic ring is substituted with 1-4 heteroatoms independently selected from O, S and N; and 6-membered heteroaryl groups in which the heteroaromatic ring is substituted with 1-3 (e.g. 1-2) nitrogen atoms; 9-membered bicyclic heteroaryl groups in which the heteroaromatic system is substituted with 1-4 heteroatoms independently selected from O, S and N; 10-membered bicyclic heteroaryl groups in which the heteroaromatic system is substituted with 1-4 nitrogen atoms. Specifically, heteroaryl groups may be independently selected from: pyrrole, furan, thiophene, pyrazole, imidazole, oxazole, isoxazole, triazole, oxadiazole, thiodiazole, tetrazole; pyridine, pyridazine, pyrimidine, pyrazine, triazine, indole, isoindole, benzofuran, isobenzofuran, benzothiophene, indazole, benzimidazole, benzoxazole, benzthiazole, benzisoxazole, purine, quinoline, isoquinoline, cinnoline, quinazoline, quinoxaline, pteridine, phthalazine, naphthyridine. In some embodiment, the heteroaryl group or 5-membered heteroaryl group is not tetrazole.

In an embodiment, applicable to any of the above aspects, the heteroaryl, phenyl and benzyl groups are optionally substituted with from 1 to 4 groups independently selected at each occurrence from: halo, nitro, cyano, hydroxyl, amino, CO₂H, CO₂—(C₁-C₄alkyl), C(O)H, C₁-C₄-alkyl, C₁-C₄ haloalkyl, C₁-C₄ alkoxy, and C₁-C₄ haloalkoxy.

In an embodiment, applicable to any of the above aspects alkyl groups and haloalkyl groups are optionally substituted with from 1 to 3 groups selected at each occurrence from oxo, imino, oximo, halo, nitro, cyano, hydroxyl, amino, CO₂H, CO₂—(C₁-C₄alkyl), C(O)H, C₁-C₄-alkyl, C₁-C₄ haloalkyl, C₁-C₄ alkoxy, and C₁-C₄ haloalkoxy.

If appropriate, the compounds according to the invention can, at certain concentrations or application rates, be used as herbicides, insect repellents and insecticides.

Depending on their particular physical and/or chemical properties, the active compounds of the invention can be converted into the customary formulations, such as solutions, emulsions, suspensions, powders, foams, pastes, granules, aerosols, microencapsulations in polymeric substances and in coating materials for seed, and also ULV cold and warm fogging formulations.

The active compounds can be used as such, in the form of their formulations or in the use forms prepared therefrom, such as ready-to-use solutions, emulsions, water- or oil-based suspensions, powders, wettable powders, pastes, soluble powders, dusts, soluble granules, granules for broadcasting, suspoemulsion concentrates, natural substances impregnated with active compound, synthetic substances impregnated with active compound, fertilizers and also microencapsulations in polymeric substances. Application is carried out in a customary manner, for example by watering, spraying, atomizing, broadcasting, dusting, foaming, spreading, etc. It is furthermore possible to apply the active compounds by the ultra-low volume method or to inject the preparation of active compound or the active compound itself into the soil. It is also possible to treat the seed of the plants.

These formulations are produced in a known manner, for example by mixing the active compounds with extenders, that is liquid solvents and/or solid carriers, optionally with the use of surfactants, that is emulsifiers and/or dispersants and/or foam-formers. The formulations are prepared either in suitable plants or else before or during the application.

Suitable for use as auxiliaries are substances which are suitable for imparting to the composition itself and/or to preparations derived therefrom (for example spray liquors, seed dressings) particular properties such as certain technical properties and/or also particular biological properties. Typical suitable auxiliaries are: extenders, solvents and carriers.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water.

Suitable solid carriers are: for example, ammonium salts and ground natural minerals such as kaolins, clays, talc, chalk, quartz, attapulgite, montmorillonite or diatomaceous earth, and ground synthetic minerals, such as finely divided silica, alumina and silicates; suitable solid carriers for granules are: for example, crushed and fractionated natural rocks such as calcite, marble, pumice, sepiolite and dolomite, and also synthetic granules of inorganic and organic meals, and granules of organic material such as paper, sawdust, coconut shells, maize cobs and tobacco stalks; suitable emulsifiers and/or foam-formers are: for example, nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; suitable dispersants are nonionic and/or ionic substances, for example from the classes of the alcohol-POE and/or -POP ethers, acid and/or POP-POE esters, alkylaryl and/or POP-POE ethers, fat- and/or POP-POE adducts, POE- and/or POP-polyol derivatives, POE- and/or POP-sorbitan- or -sugar adducts, alkyl or aryl sulphates, alkyl- or arylsulphonates and alkyl or aryl phosphates or the corresponding PO-ether adducts. Furthermore, suitable oligo- or polymers, for example those derived from vinylic monomers, from acrylic acid, from EO and/or PO alone or in combination with, for example, (poly)alcohols or (poly)amines. It is also possible to employ lignin and its sulphonic acid derivatives, unmodified and modified celluloses, aromatic and/or aliphatic sulphonic acids and their adducts with formaldehyde.

Tackifiers such as carboxymethylcellulose and natural and synthetic polymers in the form of powders, granules or lattices, such as gum arabic, polyvinyl alcohol and polyvinyl acetate, as well as natural phospholipids such as cephalins and lecithins, and synthetic phospholipids, can be used in the formulations.

Further additives may be mineral and vegetable oils. It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc. Other possible additives are perfumes, mineral or vegetable, optionally modified oils, waxes and nutrients (including trace nutrients), such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present.

The formulations generally comprise between 0.01 and 98% by weight of active compound, preferably between 0.1 and 95% and particularly preferably between 0.5 and 90%.

The active compounds according to the invention, as such or in their formulations, can also be used as a mixture with known fungicides, bactericides, acaricides, nematicides, or insecticides, for example, to improve the activity spectrum or prevent the development of resistance.

A mixture with other known active compounds such as herbicides, or with fertilizers and growth regulators, safeners or semiochemicals is also possible.

Exemplary application rates of the active compounds according to the invention are: when treating leaves: from 0.1 to 10 000 g/ha, preferably from 10 to 1000 g/ha, particularly preferably from 50 to 300 g/ha (when the application is carried out by watering or dripping, it is even possible to reduce the application rate, especially when inert substrates such as rock wool or perlite are used); when treating seed: from 2 to 200 g per 100 kg of seed, preferably from 3 to 150 g per 100 kg of seed, particularly preferably from 2.5 to 25 g per 100 kg of seed, very particularly preferably from 2.5 to 12.5 g per 100 kg of seed; when treating the soil: from 0.1 to 10 000 g/ha, preferably from 1 to 5000 g/ha.

The compositions according to the invention are suitable for protecting any plant variety which is employed in agriculture, in the greenhouse, in forests or in horticulture and, in particular, cereals (such as wheat, barley, rye, millet and oats), maize, cotton, soya beans, rice, potatoes, sunflowers, beans, coffee, beet (for example sugar beet and fodder beet), peanuts, vegetables (such as tomatoes, cucumbers, onions and lettuce), lawns and ornamental plants.

The active compounds of the invention, in combination with good plant tolerance and favourable toxicity to warm-blooded animals and being tolerated well by the environment, are suitable for protecting plants and plant organs, for increasing the harvest yields, for improving the quality of the harvested material and for controlling animal pests, in particular insects, arachnids, helminths, nematodes and mollusks, which are encountered in agriculture, in horticulture, in animal husbandry, in forests, in gardens and leisure facilities, in the protection of stored products and of materials, and in the hygiene sector. They may be preferably employed as crop protection agents. They are active against normally sensitive and resistant species and against all or some stages of development. The abovementioned pests include: from the order of the Anoplura (Phthiraptera), for example, Damalinia spp., Haematopinus spp., Linognathus spp., Pediculus spp., Trichodectes spp; from the class of the Arachnida, for example, Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici; from the class of the Bivalva, for example, Dreissena spp; from the order of the Chilopoda, for example, Geophilus spp., Scutigera spp; from the order of the Coleoptera, for example, Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnostema consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogoderma spp., Tychius spp., Xylotrechus spp., Zabrus spp; from the order of the Collembola, for example, Onychiurus armatus; from the order of the Dermaptera, for example, Forficula auricularia; from the order of the Diplopoda, for example, Blaniulus guttulatus; from the order of the Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp., Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa, Wohlfahrtia spp; from the class of the Gastropoda, for example, Anion spp., Biomphalaria spp., Bulinus spp., Deroceras spp., Galba spp., Lymnaea spp., Oncomelania spp., Succinea spp; from the class of the helminths, for example, Ancylostoma duodenale, Ancylostoma ceylanicum, Acylostoma braziliensis, Ancylostoma spp., Ascaris lumbricoides, Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichiura, Wuchereria bancrofti.

When used as insecticides, the active compounds according to the invention can furthermore be present in their commercially available formulations and in the use forms, prepared from these formulations, as a mixture with inhibitors which reduce degradation of the active compound after use in the environment of the plant, on the surface of parts of plants or in plant tissues. The active compound content of the use forms prepared from the commercially available formulations can vary within wide limits. The active compound concentration of the use forms can be from 0.00000001 to 95% by weight of active compound, preferably between 0.00001 and 1% by weight. The compounds are employed in a customary manner appropriate for the use forms.

The active compounds according to the invention act not only against plant, hygiene and stored product pests, but also in the veterinary medicine sector against animal parasites (ecto- and endoparasites), such as hard ticks, soft ticks, mange mites, leaf mites, flies (biting and licking), parasitic fly larvae, lice, hair lice, feather lice and fleas. These parasites include: from the order of the Anoplurida, for example, Haematopinus spp., Linognathus spp., Pediculus spp., Phtirus spp., Solenopotes spp; from the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp., Felicola spp; diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp; from the order of the Siphonapterida, for example, Pulex spp., Ctenocephalides spp., Xenopsylla spp., Ceratophyllus spp; from the order of the Heteropterida, for example, Cimex spp., Triatoma spp., Rhodnius spp., Panstrongylus spp; from the order of the Blattarida, for example, Blatta orientalis, Periplaneta americana, Blattela germanica, Supella spp; from the subclass of the Acari (Acarina) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp., Varroa spp; from the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp. Each compound of the invention may have activity against one or more than one of the above organisms.

The active compounds according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reductions in productivity (for meat, milk, wool, hides, eggs, honey etc.) should be diminished, so that more economic and easier animal husbandry is possible by use of the active compounds according to the invention.

The active compounds according to the invention are used in the veterinary sector and in animal husbandry in a known manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants, by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for cattle, poultry, pets and the like, the active compounds of the invention can be used as formulations (for example powders, emulsions, free-flowing compositions), which comprise the active compounds in an amount of 1 to 80% by weight, directly or after 100- to 10 000-fold dilution, or they can be used as a chemical bath.

It has furthermore been found that the compounds according to the invention also have a strong insecticidal action against insects which destroy industrial materials.

The following insects may be mentioned as examples and as preferred—but without any limitation: Beetles, such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec. Tryptodendron spec. Apate monachus, Bostrychus capucins, Heterobostrychus brunneus, Sinoxylon spec. Dinoderus minutus;

Hymenopterons, such as Sirex juvencus, Urocerus gigas, Urocerus gigas taignus, Urocerus augur; Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus; Bristletails, such as Lepisma saccharina. Each compound of the invention may have activity against one or more than one of the above organisms.

Industrial materials in the present connection are to be understood as meaning non-living materials, such as, preferably, plastics, adhesives, sizes, papers and cardboards, leather, wood and processed wood products and coating compositions.

In domestic, hygiene and stored-product protection, the active compounds are also suitable for controlling animal pests, in particular insects, arachnids and mites, which are found in enclosed spaces such as, for example, dwellings, factory halls, offices, vehicle cabins and the like. They can be employed alone or in combination with other active compounds and auxiliaries in domestic insecticide products for controlling these pests. They are active against sensitive and resistant species and against all developmental stages. These pests include: from the order of the Scorpionidea, for example, Buthus occitanus; from the order of the Acarina, for example, Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae; from the order of the Araneae, for example, Aviculariidae, Araneidae; from the order of the Opiliones, for example, Pseudoscorpiones chelifer, Pseudoscorpiones cheiridium, Opiliones phalangium; from the order of the Isopoda, for example, Oniscus asellus, Porcellio scaber; from the order of the Diplopoda, for example, Blaniulus guttulatus, Polydesmus spp; from the order of the Chilopoda, for example, Geophilus spp; from the order of the Zygentoma, for example, Ctenolepisma spp., Lepisma saccharina, Lepismodes inquilinus; from the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa; from the order of the Saltatoria, for example, Acheta domesticus; from the order of the Dermaptera, for example, Forficula auricularia; from the order of the Isoptera, for example, Kalotermes spp., Reticulitermes spp; from the order of the Psocoptera, for example, Lepinatus spp., Liposcelis spp; from the order of the Coleoptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum; from the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa; from the order of the Lepidoptera, for example, Achroia grisella, Galleria mellonella, Plodia interpunctella, Tinea cloacella, Tinea pellionella, Tineola bisselliella; from the order of the Siphonaptera, for example, Ctenocephalides canis, Ctenocephalides felis, Pulex irritans, Tunga penetrans, Xenopsylla cheopis; from the order of the Hymenoptera, for example, Camponotus herculeanus, Lasius fuliginosus, Lasius niger, Lasius umbratus, Monomorium pharaonis, Paravespula spp., Tetramorium caespitum; from the order of the Anoplura, for example, Pediculus humanus capitis, Pediculus humanus corporis, Pemphigus spp., Phylloera vastatrix, Phthirus pubis; from the order of the Heteroptera, for example, Cimex hemipterus, Cimex lectularius, Rhodinus prolixus, Triatoma infestans. Each compound of the invention may have activity against one or more than one of the above organisms.

In the field of household insecticides, they are used alone or in combination with other suitable active compounds, such as phosphoric esters, carbamates, pyrethroids, neonicotinoids, growth regulators or active compounds from other known classes of insecticides. They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

Many of the compounds of the invention have excellent herbicidal activity against a broad spectrum of economically important mono- and dicotyledonous harmful plants. Many of the compounds of the invention are selective, having excellent herbicidal activity against monocotyledonous harmful plants but no activity or little activity against dicotyledonous crops. Other compounds of the invention are selective, having excellent herbicidal activity against dicotyledonous harmful plants but no activity or little activity against monocotyledonous crops. Difficult-to-control perennial weeds which produce shoots from rhizomes, root stocks or other perennial organs are also well controlled by the active compounds. Here, the substances can be applied, for example, by the pre-sowing method, the pre-emergence method and/or the post-emergence method, for example jointly or separately. Post-emergence application is preferred.

Specific mention may be made of some representatives of the mono- and dicotyledonous weed flora which can be controlled by the combinations according to the invention; however, this list is not to be understood as meaning a limitation to certain species.

Examples of weed species which are controlled efficiently are, from amongst the monocotyledonous weed species, Avena spp., Alopecurus spp., Brachiaria spp., Digitaria spp., Lolium spp., Echinochloa spp., Panicum spp., Phalaris spp., Poa spp., Setaria spp. and also Bromus spp. such as Bromus catharticus, Bromus secalinus, Bromus erectus, Bromus tectorum and Bromus japonicus and Cyperus species from the annual group, and, among the perennial species, Agropyron, Cynodon, Imperata and Sorghum and also perennial Cyperus species.

In the case of dicotyledonous weed species, the spectrum of action extends to genera such as, for example, Abutilon spp., Amaranthus spp., Chenopodium spp., Chrysanthemum spp., Galium spp. such as Galium aparine, Ipomoea spp., Kochia spp., Lamium spp., Matricaria spp., Pharbitis spp., Polygonum spp., Sida spp., Sinapis spp., Solanum spp., Stellaria spp., Veronica spp. and Viola spp., Xanthium spp., among the annuals, and Convolvulus, Cirsium, Rumex and Artemisia in the case of the perennial weeds.

If the combinations according to the invention are applied to the soil surface before or during germination, the weed seedlings are inhibited or prevented completely from emerging or else the weeds grow until they have reached the cotyledon stage, but then their growth stops, and, eventually, after three to four weeks have elapsed, they die completely.

If the active compounds are applied post-emergence to the green parts of the plants, growth likewise stops rapidly a very short time after the treatment, and the weed plants remain at the growth stage of the point of time of application, or they die completely after a certain time, so that in this manner competition by the weeds, which is harmful to the crop plants, is eliminated very early and in a sustained manner.

Some of the compounds of the invention are useful as insect repellents. These compounds may be formulated in such a way as to be applicable to humans, e.g. as a topical formulation with pharmaceutically acceptable excipients.

### DETAILED DESCRIPTION

### Synthesis

The compounds of the invention are based on active compounds as disclosed above. The synthetic routes to each of the parent compounds are available in the literature. These disclosures relating to the parent compounds insofar as the synthetic procedures are concerned specifically form part of the disclosure of the present invention. Whilst the compounds of the present invention may be prepared directly using standard procedures, they may sometimes more conveniently be prepared from the parent compounds by conventional synthetic procedures. In the interests of brevity, the details of these synthetic procedures are not reproduced here but it is intended that this subject matter is specifically incorporated into the disclosure of these documents by reference.

Equally, the compounds can be prepared by total or partial synthesis. Thus, conveniently, the derivatives of each parent active may in some cases be prepared directly from the respective parent active itself by reactions known to the skilled person. However, in practice the skilled person will design a suitable synthetic procedure, including convergent synthesis, to prepare a given derivative depending on its particular functionality and oxidation state. The skilled person is familiar with such procedures and these represent common general knowledge as set out in text books such as Warren “Organic Synthesis: The Disconnection Approach”; Mackie and Smith “Guidebook to Organic Chemistry”; and Clayden, Greeves, Warren and Wothers “Organic Chemistry”.

For convenience only, the derivatives of the invention may be obtained by effecting oxidation or reduction of the target functional group at an intermediate stage in the synthesis rather than at a final stage in the synthesis of the derivatives of the present invention. Where necessary, the skilled person will be aware of the need to use suitable protecting groups to protect other functionalities in the molecule from unwanted oxidation or reduction during transformation of the target functional group.

The skilled man will appreciate that adaptation of methods known in the art could be applied in the manufacture of the compounds of the present invention.

For example, the skilled person will be immediately familiar with standard textbooks such as “Comprehensive Organic Transformations—A Guide to Functional Group Transformations”, R C Larock, Wiley-VCH (1999 or later editions), “March's Advanced Organic Chemistry—Reactions, Mechanisms and Structure”, M B Smith, J. March, Wiley, (5th edition or later) “Advanced Organic Chemistry, Part B, Reactions and Synthesis”, F A Carey, R J Sundberg, Kluwer Academic/Plenum Publications, (2001 or later editions), “Organic Synthesis—The Disconnection Approach”, S Warren (Wiley), (1982 or later editions), “Designing Organic Syntheses” S Warren (Wiley) (1983 or later editions), “Guidebook To Organic Synthesis” R K Mackie and D M Smith (Longman) (1982 or later editions), etc., and the references therein as a guide.

The skilled chemist will exercise his judgement and skill as to the most efficient sequence of reactions for synthesis of a given target compound and will employ protecting groups as necessary. This will depend inter alia on factors such as the nature of other functional groups present in a particular substrate. Clearly, the type of chemistry involved will influence the choice of reagent that is used in the said synthetic steps, the need, and type, of protecting groups that are employed, and the sequence for accomplishing the protection/deprotection steps. These and other reaction parameters will be evident to the skilled person by reference to standard textbooks and to the examples provided herein.

Sensitive functional groups may need to be protected and deprotected during synthesis of a compound of the invention. This may be achieved by conventional methods, for example as described in “Protective Groups in Organic Synthesis” by T W Greene and P G M Wuts, John Wiley & Sons Inc (1999), and references therein.

Compounds of the invention containing one or more asymmetric carbon atoms can exist as two or more stereoisomers. Where a compound of the invention contains a double bond such as a C═C or C═N group, geometric cis/trans (or Z/E) isomers are possible. Where structural isomers are interconvertible via a low energy barrier, tautomeric isomerism (‘tautomerism’) can occur. This can take the form of proton tautomerism in compounds of the invention containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of the invention, including compounds exhibiting more than one type of isomerism, and mixtures of one or more thereof. Also included are acid addition or base salts wherein the counter ion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers when necessary include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

The activity of the compounds of the present invention can be assessed by a variety of in silico, in vitro and in vivo assays. In silico analysis of a variety of compounds has been demonstrated to be predictive of ultimate in vitro and even in vivo activity.

The present invention also includes the synthesis of all environmentally acceptable isotopically-labelled compounds of formulae (I) to (XXIII) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Isotopically-labelled compounds can generally be prepared by conventional techniques known to those skilled in the art or by processes analogous to those described using an appropriate isotopically-labelled reagent in place of the non-labelled reagent previously employed.

Throughout this specification these abbreviations have the following meanings:

TPAP—tetrapropylammonium perruthenate

### NMO—N-methylmorpholine-N-oxide

### DMF—N, N-dimethylformamide

DCM—dichloromethane
TFA—trifluoroacetic acid
LDA—lithium diisopropylamide
MOM—methoxymethyl
HMDS—hexamethyldisilazide
MCPBA—meta-chloroperbenzoic acid
MCBA—meta-chlorobenzoic acid
TLC—thin layer chromatography
DMAP—N,N-dimethyl-4-aminopyridine
DCC—N,N′-dicyclohexylcarbodiimide
DIBAL-H—diisobutylaluminium hydride
BOC—tert-butyl carbonate

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### Example 1

### Mesosulfuron Derivatives 8-12

Mesosulfuron methyl ester 6 (the form in which mesosulfuron is typically administered) can be synthesised using the following known sequence of reactions:

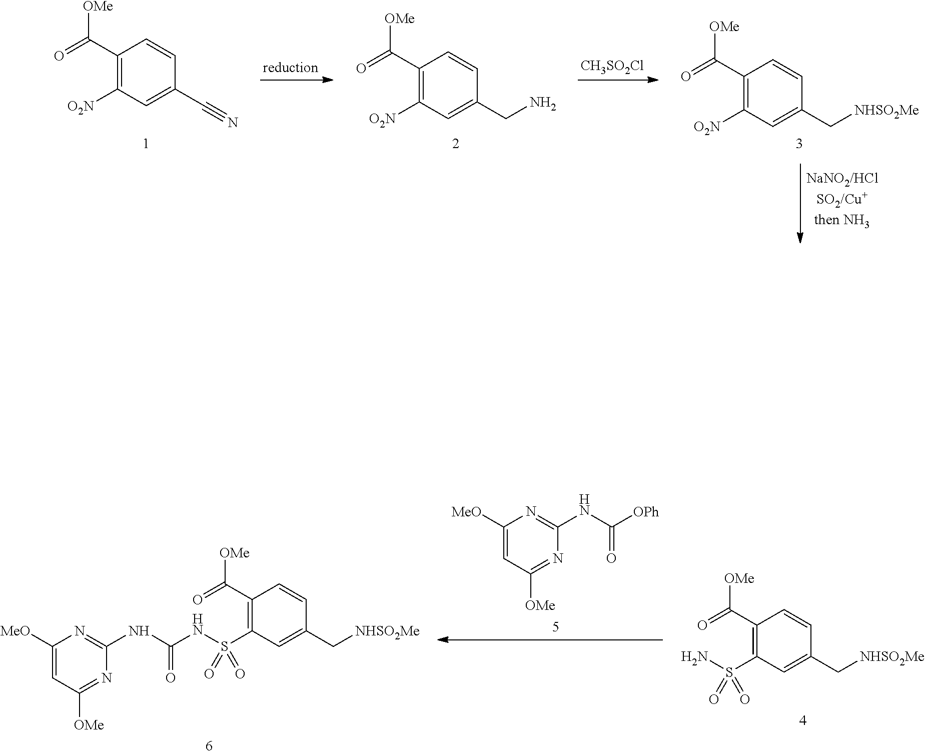

Mesosulfuron derivatives 8-12 can be made from mesosulfuron 7 or mesosulfuron methyl ester 6. Mesosulfuron aldehyde 9 can be prepared from the acid by conversion of the acid to the Weinreb amide. This will typically be done by mixing the acid with the Weinreb amine and an activating agent (e.g. DCC) and a nucleophillic catalyst (e.g. DMAP). Alternatively this can be done by generating the acid chloride (using a chlorinating agent such as oxaloyl chloride or thionyl chloride) and subsequently treating the acid chloride with the Weinreb amine in the presence of a base (such as pyridine, which may also be the solvent). Once formed the Weinreb amide can be reduced with any suitable reducing agent (e.g. DIBAL-H). Alcohol 8 can be prepared from the acid 7 by reduction. An appropriate reductant would be LiAlH₄, in which case the reaction is suitably conducted in ether. Another alternative method of forming the aldehyde 9 is to oxidise the alcohol 8 using, for example, a Swern oxidation or TPAP/NMO or Dess-Martin periodinane under standard conditions. Alcohol 8 which can be acetylated under standard conditions. One option would be to use AcCl or Ac₂O in the presence of a base (e.g. pyridine, which may also be the solvent, or triethylamine in which case the solvent may be DCM) and optionally a nucleophillic catalyst (e.g. DMAP). The mesosulfuron acetals 11a-b can be accessed by treating mesosulfuron aldehyde 9 with an alcohol in the presence of an acid. It may be preferable to include a method of removing water from the reaction (e.g. using molecular sieves or a Dean-Stark apparatus). The mesosulfuron oximes 10a-c can be accessed by condensing mesosulfuron aldehyde 9 with an appropriately substituted hydroxylamine. The reaction can be carried out in the present of an acid. A condensation/cyclisation reaction between aldehyde 9, a source of ammonia (e.g. NH₄OAc), and oxaldehyde or an oxaldehyde equivalent can provide imidazole 12.

Alternatively, an aldehyde may have to be introduced at an earlier stage of the synthesis e.g. before the mesylation step to form the aldehyde equivalent of 2 or before the aminosulfonation step to form the aldehyde equivalent of 3 or before the coupling step to form the aldehyde equivalent of 4. In this case the aldehyde would be introduced and the resulting compound subjected to the same reaction steps described in the scheme above to form aldehyde 9.

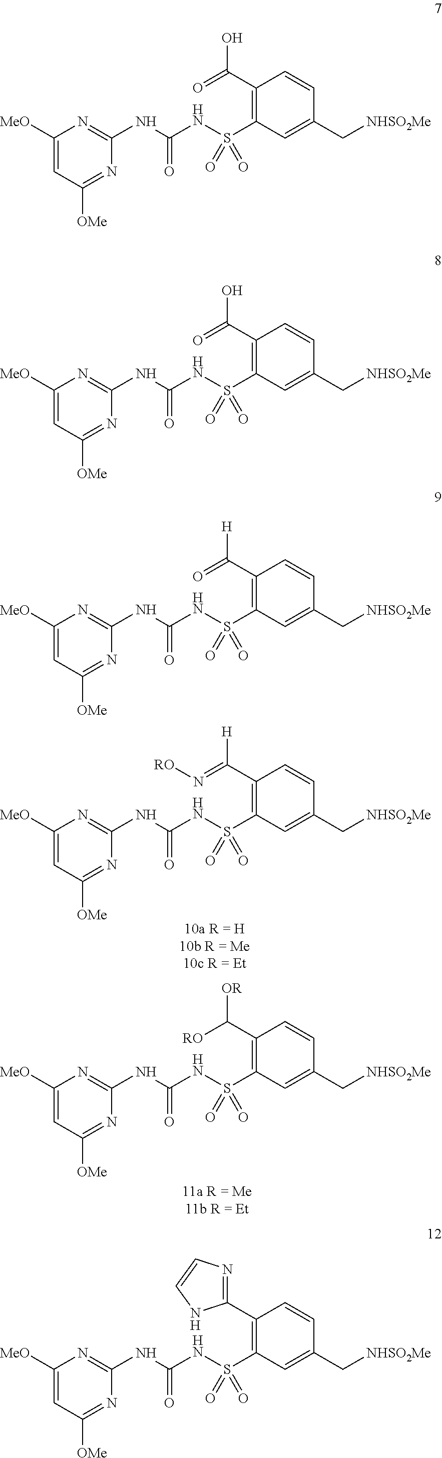

### Example 2

### Cyhalothrin Derivatives 13-20

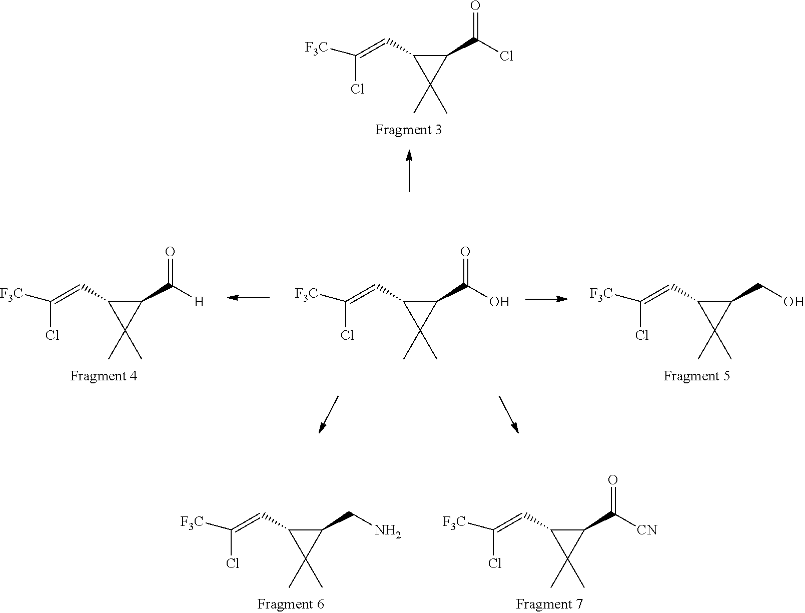 [SMILES: CC1(C)[C@H](/C=C(\Cl)C(F)(F)F)[C@H]1C(=O)Cl ; CC1(C)[C@H](C=O)[C@H]1/C=C(\Cl)C(F)(F)F ; CC1(C)[C@H](/C=C(\Cl)C(F)(F)F)[C@H]1C(=O)O ; CC1(C)[C@H](/C=C(\Cl)C(F)(F)F)[C@H]1CO ; CC1(C)[C@H](/C=C(\Cl)C(F)(F)F)[C@H]1CN ; CC1(C)[C@H](/C=C(\Cl)C(F)(F)F)[C@H]1C(=O)C#N]

Fragment 1 can be obtained by hydrolysis (using e.g. NaOH) of cyhalothrin. It is possible to derive Fragments 3-7 from Fragment 1 (the carboxylic acid shown in the scheme above). Reaction of Fragment 1 with a chlorinating agent (e.g. oxaloyl chloride or thionyl chloride) gives the acid chloride Fragment 3. Reduction of Fragment 1 with a reducing agent (e.g. LiAlH₄) gives alcohol Fragment 5 which can then be oxidised (Swern, Dess-Martin etc) to give aldehyde Fragment 4. Amine Fragment 6 can be accessed from a number of alternate methods. From Fragment 5, halide exchange and subsequent azide introduction and reduction is one approach, or halide exchange and Gabriel synthesis another. Alternatively, reductive amination of Fragment 4 under the appropriate conditions would lead to the desired structure. Treatment of Fragment 4 with a cyanide source (e.g. NaCN) gives Fragment 7.

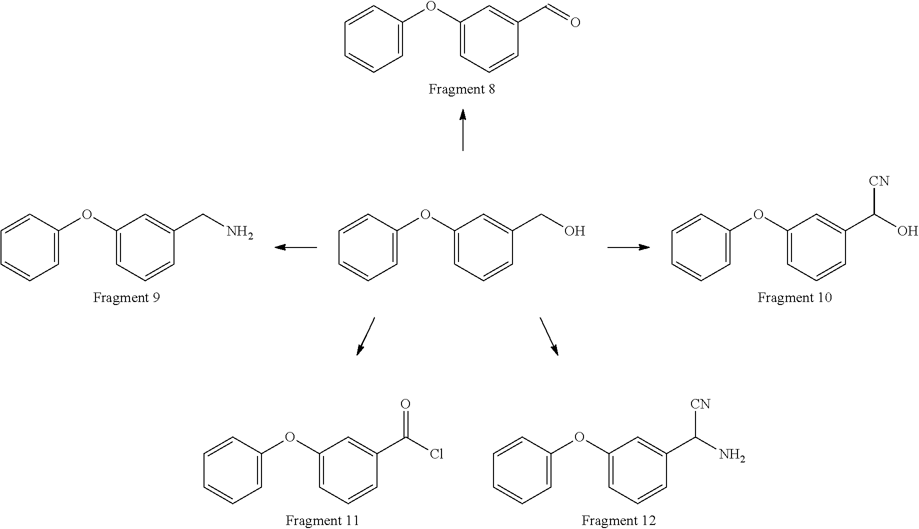 [SMILES: O=Cc1cccc(Oc2ccccc2)c1 ; NCc1cccc(Oc2ccccc2)c1 ; OCc1cccc(Oc2ccccc2)c1 ; N#CC(O)c1cccc(Oc2ccccc2)c1 ; O=C(Cl)c1cccc(Oc2ccccc2)c1 ; N#CC(N)c1cccc(Oc2ccccc2)c1]

A similar set of fragments can be envisaged for Fragment 2 (the alcohol depicted in the centre of the scheme above). Fragment 2 is commercially available, as are Fragments 8 and 9 though it should be possible to access these from Fragment 2 using the same transformations as detailed for Fragments 4 and 6 above. Treatment of Fragment 8 with a cyanide source (e.g. NaCN) gives Fragment 10 while subsequent conversion of the alcohol in fragment 10 to the amine (using the same transformations as given for Fragment 6) will give Fragment 12. Oxidation of Fragment 2 to the carboxylic acid (using e.g. KMnO₄) and treatment with a chlorinating agent (e.g. oxaloyl chloride or thionyl chloride) gives Fragment 11.

The fragments above can be combined to generate derivatives 13-20 below using coupling transformations which will be familiar to the skilled in the art. The couplings are as follows: 13—fragments 3 and 2 coupled using an esterification reaction (optionally in the presence of a base); 14—fragments 5 and 11 coupled using an esterification reaction (optionally in the presence of a base); 15—fragments 3 and 10 coupled using an esterification reaction (optionally in the presence of a base); 16—fragments 7 and 11 coupled using an esterification reaction (optionally in the presence of a base); 17—fragments 6 and 11 coupled using an amide bond forming reaction (optionally in the presence of a base); 18—fragments 4 and 12 coupled using a condensation reaction (in the presence of a base or an acid); 19—fragments 3 and 12 coupled using an amide bond forming reaction (optionally in the presence of a base); 20—fragments 4 and 12 coupled using reductive amination reaction (e.g. using NaBH(OAc)₃).

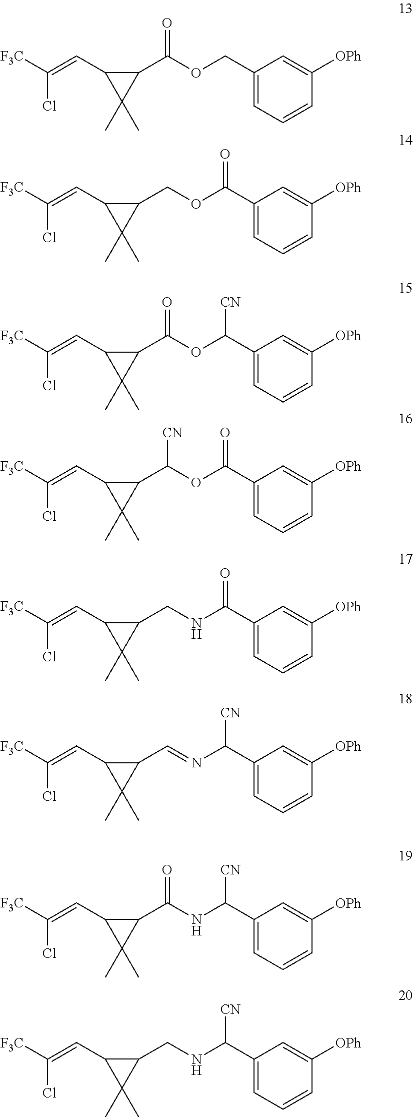

### Example 3

### Permethrin, Deltamethrin Derivatives

Permethrin, deltamethrin derivatives can be made analogously to the cyhalothrin derivatives described in Example 2.

### Example 4

### Ethyl Fenoxaprop 23, Fluazifop 25, Clodinafop 26

Fenoxaprop, fluazifop and clodinafop are made from an α-halo propionic acid, hydroquinone and the 2-chlorobenzoxazoles or 2-chloropyridines. The order of reaction steps is not important as illustrated by the following schemes (taken from GB1548847) detailing the synthesis of ethyl fenoxaprop 23:

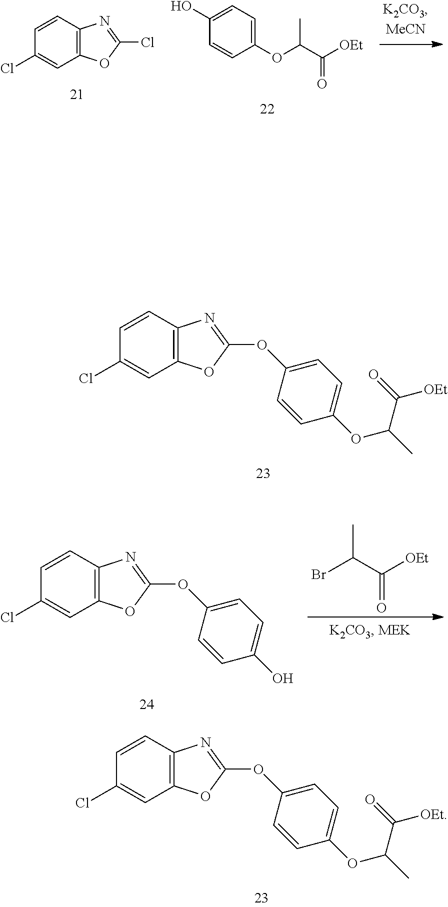

The active compound in each case is the acid. This can be obtained from the ethyl ester by hydrolysis, e.g. using a base (e.g. NaOH).

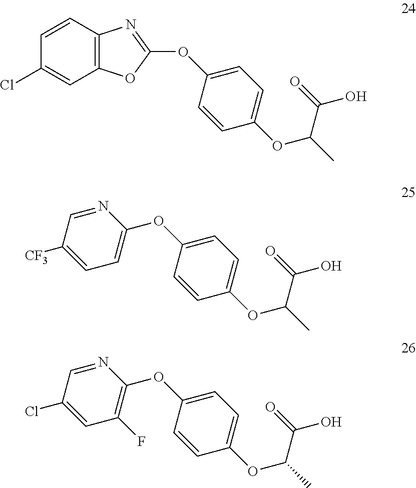 [SMILES: CC(Oc1ccc(Oc2nc3ccc(Cl)cc3o2)cc1)C(=O)O ; CC(Oc1ccc(Oc2ccc(C(F)(F)F)cn2)cc1)C(=O)O ; C[C@H](Oc1ccc(Oc2ncc(Cl)cc2F)cc1)C(=O)O]

### Example 5

### Fluazifop Alcohol 27 and Aldehyde 28

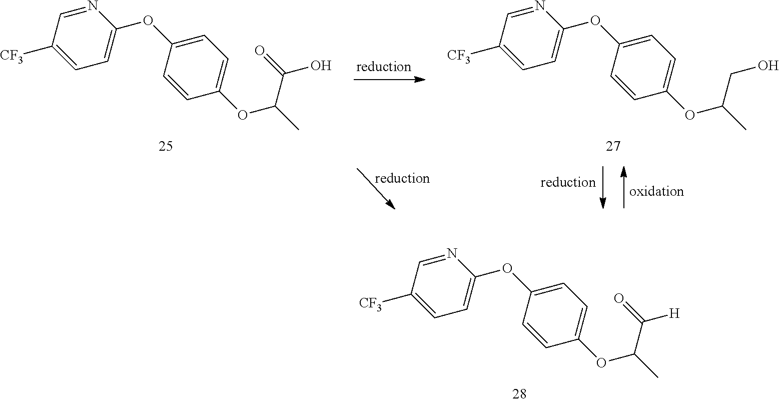 [SMILES: CC(Oc1ccc(Oc2ccc(C(F)(F)F)cn2)cc1)C(=O)O ; CC(CO)Oc1ccc(Oc2ccc(C(F)(F)F)cn2)cc1 ; CC(C=O)Oc1ccc(Oc2ccc(C(F)(F)F)cn2)cc1]

Fluazifop aldehyde 28 can be prepared from the acid by conversion of the acid to the Weinreb amide. This will typically be done by mixing the acid with the Weinreb amine and an activating agent (e.g. DCC) and a nucleophillic catalyst (e.g. DMAP). Alternatively this can be done by generating the acid chloride (using a chlorinating agent such as oxaloyl chloride or thionyl chloride) and subsequently treating the acid chloride with the Weinreb amine in the presence of a base (such as triethylamine or pyridine, which may also be the solvent). Once formed the Weinreb amide can be reduced with any suitable reducing agent (e.g. DIBAL-H).

Alcohol 27 can be prepared from fluazifop 25 by reduction. An appropriate reductant would be LiAlH₄, in which case the reaction is suitably conducted in ether. Another alternative method of forming the aldehyde 28 is to oxidise the alcohol 27 using, for example, a Swern oxidation or TPAP/NMO or Dess-Martin periodinane under standard conditions. The alcohols and aldehydes of clodinafop and fenoxaprop can be formed from clodinafop 28 and fenoxaprop 25 analogously.

### Example 6

### Clodinafop Oximes and Acetals 30a-31b

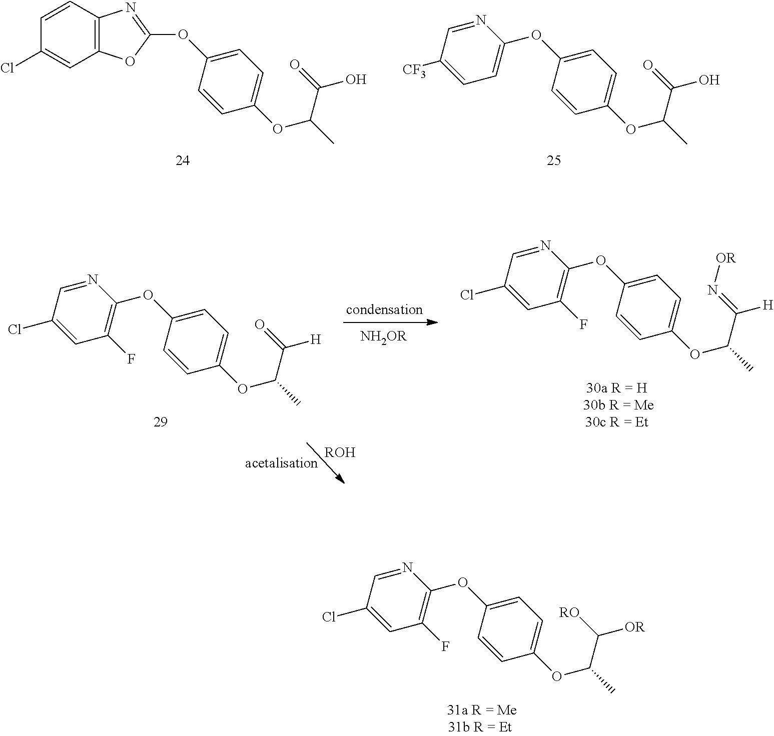

The clodinafop oximes 30a-c can be accessed by condensing clodinafop aldehyde 29 with an appropriately substituted hydroxylamine. The reaction can be carried out in the presence of an acid. The clodinafop acetals 31a-b can be accessed by treating clodinafop aldehyde 29 with an alcohol in the presence of an acid. It may be preferable to include a method of removing water from the reaction (e.g. using molecular sieves or a Dean-Stark apparatus). Fluazifop and fenoxaprop oximes and acetals can be synthesised from the corresponding aldehydes using analogous methods.

### Example 7

### Fenoxaprop Acetate 33

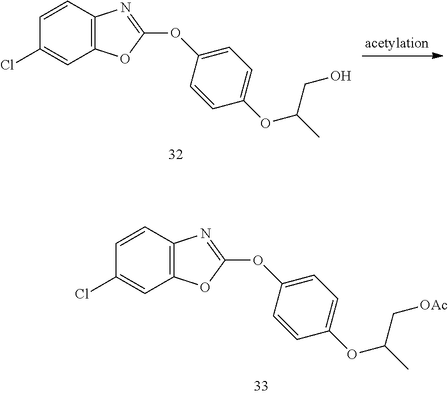 [SMILES: CC(CO)Oc1ccc(Oc2nc3ccc(Cl)cc3o2)cc1 ; CC(=O)OCC(C)Oc1ccc(Oc2nc3ccc(Cl)cc3o2)cc1]

Fenoxaprop alcohol 32 can be acetylated under standard conditions. One option would be to use AcCl or Ac₂O in the presence of a base (e.g. pyridine, which may also be the solvent, or triethylamine in which case the solvent may be DCM) and optionally a nucleophillic catalyst (e.g. DMAP). Clodinafop and fenoxaprop acetates can be synthesised from the corresponding alcohols using analogous methods.

### Example 8

### Icaridin 38

A synthesis of icaridin 38 using phosgene is described in U.S. Pat. No. 4,900,834. An alternative synthesis using carbonyl di-imidazole 35 is described in the scheme below.

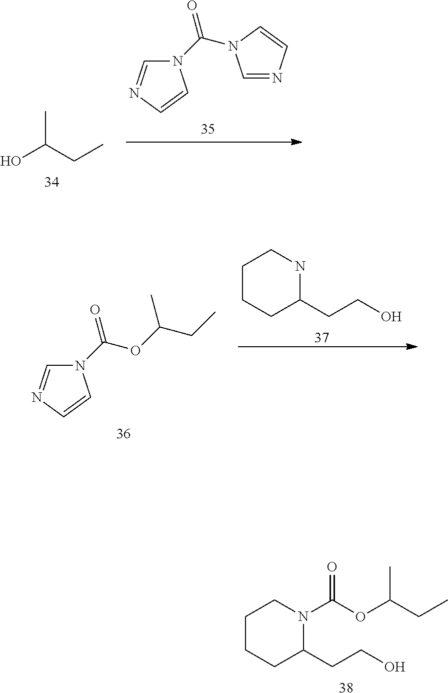

### Example 9

### Icaridin Aldehyde 39

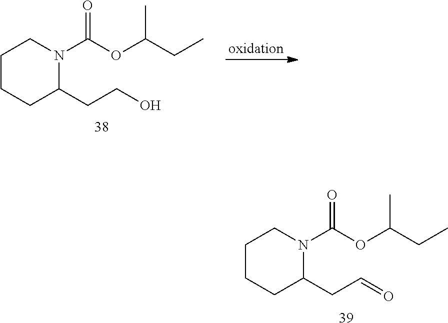

Icaridin 38 can be oxidised to icaridin aldehyde 39 using appropriate oxidising conditions e.g. a Swern oxidation, using TPAP/NMO or using Dess-Martin periodinane under standard conditions.

### Example 10

### Icaridin Oximes 40a-c

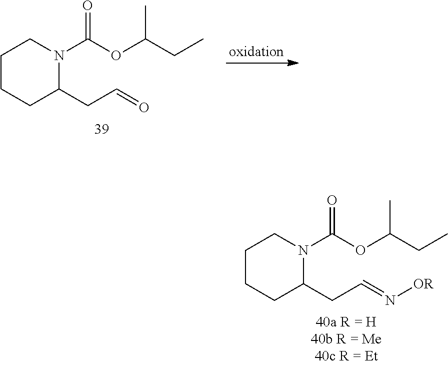

The icaridin oximes 40a-c can be accessed by condensing icaridin aldehyde 39 with an appropriately substituted hydroxylamine. The reaction can be carried out in the presence of an acid or a base.

### Example 11

### Icaridin Acid and Esters 41-62b

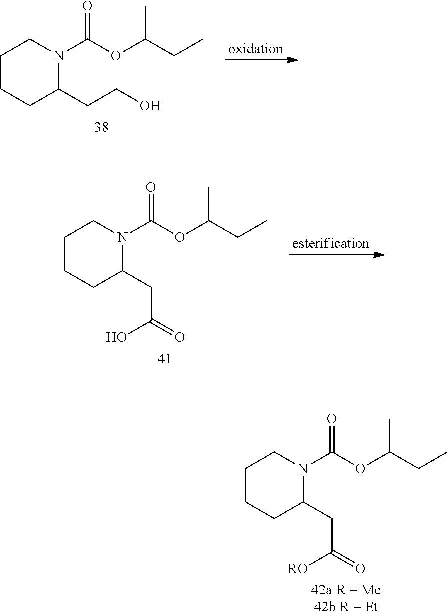

Icaridin 38 can be oxidised to the acid 41. This can be achieved using an appropriate oxidising agent (e.g. KMnO₄). The acid can be converted into the esters 42a-b by treatment with the corresponding alcohols optionally in the presence of an acid (e.g. AcCl in the alcohol). Alternatively the methyl ester 42a can be formed using a methylating agent (e.g. diazomethane or trimethylsilyldiazomethane).

### Example 12

### Cyantraniliprole Ethyl Amide 47

WO2004067528 describes the synthesis of cyantraniliprole from acid 43 and acid 44. The syntheses of acids 43 and 44 are also described in WO2004067528. The ethyl amide 47 can be made be using ethylamine rather than methylamine in the final step of the synthesis as shown below.

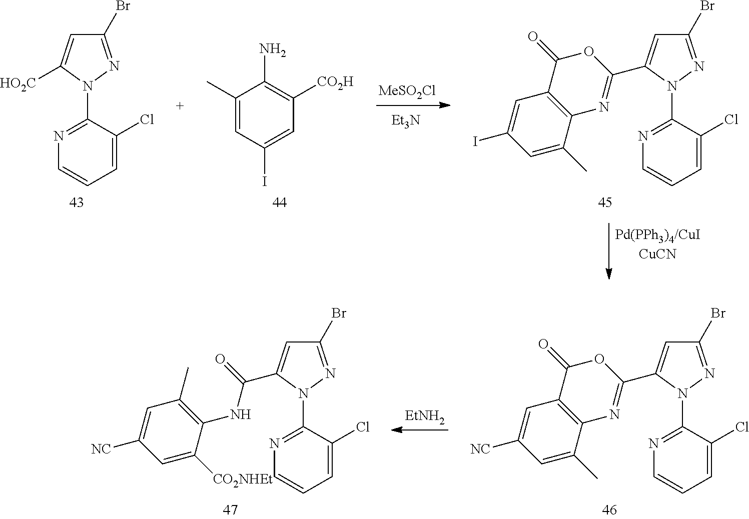

### Example 13

### Cyantraniliprole Aldehyde 50 and Oximes 51a-c

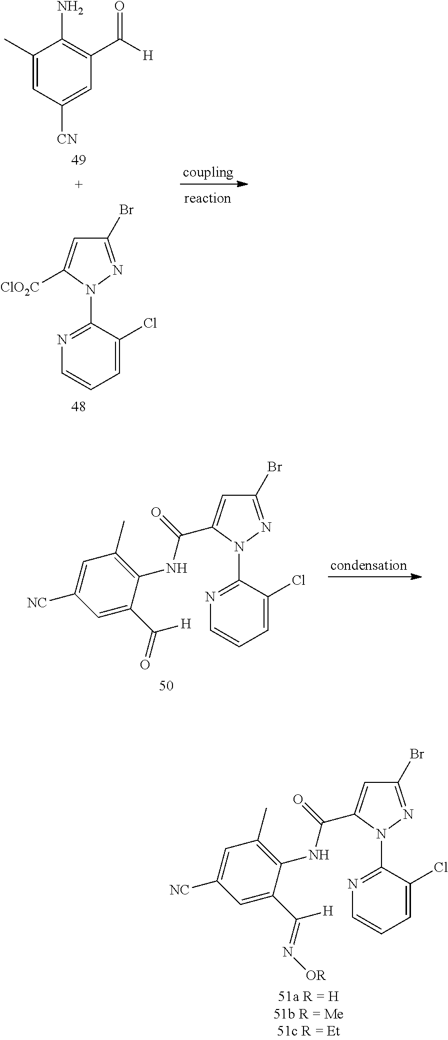

Manipulation of acids 43 and 44 can provide aldehyde 49 and acid chloride 48, which can be coupled under amide bond forming conditions (in the presence of a base) to provide aldehyde 50. The aldehyde can optionally be protected during the coupling step e.g. as an acetal.

The cyantraniliprole oximes 51a-c can then be accessed by condensing cyantraniliprole aldehyde 50 with an appropriately substituted hydroxylamine. The reaction can be carried out in the presence of an acid.

### Example 14

### Cyantraniliprole Imine 54

Manipulation of acids 43 and 44 can provide aldehyde 53 and amide 52, which can be coupled in a condensation reaction to provide imine 54. This can be achieved in acid in or basic conditions. It may be preferable to provide a means for removing water such as molecular sieves (this is particularly appropriate when the reaction is performed in the presence of a base). Alternatively, if the base is sodium carbonate, which may itself be a drying agent. The means may be a Dean Stark apparatus (this is particularly appropriate when the reaction is performed in the presence of an acid).

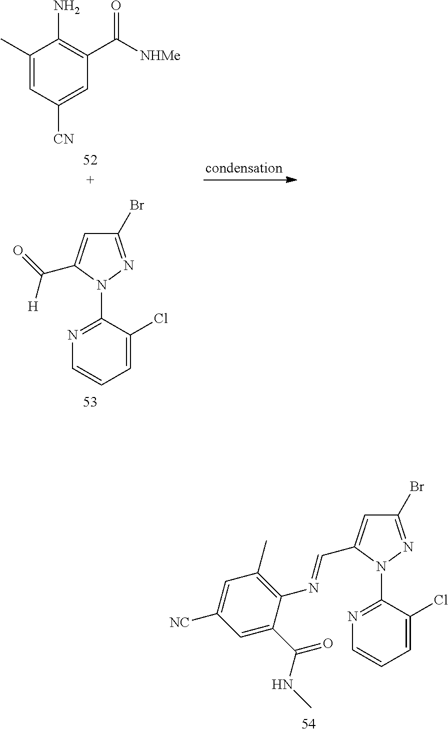

### Example 15

### [Cyano-(3-phenoxyphenyl)methyl] 3-[(Z)-2-chloro-3,3,3-trifluoro-prop-1-enyl]-2,2-dimethyl-cyclopropanecarboxylate 15

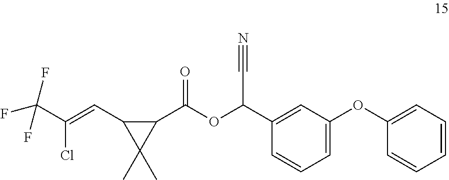 [SMILES: CC1(C)C(/C=C(\Cl)C(F)(F)F)C1C(=O)OC(C#N)c1cccc(Oc2ccccc2)c1]

A solution of 3-[(Z)-2-chloro-3,3,3-trifluoro-prop-1-enyl]-2,2-dimethyl-cyclopropanecarbonyl chloride (210 mg, 1.1 eq) in toluene (6 mL) was added dropwise to a solution of 2-hydroxy-2-(3-phenoxyphenyl)acetonitrile (165 mg, 1 eq) and pyridine (59 μL, 1 eq) in toluene (5 mL). The reaction mixture was stirred overnight at room temperature after which time TLC analysis showed the reaction had gone to completion. The reaction mixture was diluted with ethyl acetate (15 mL) and washed with water (2×10 mL) and brine (10 mL) before being dried over MgSO₄ and the solvent removed in vacuo. The residue was purified by flash chromatography (solvent 95:5 hexane/ethyl acetate) to afford the product as a clear oil (204 mg, 62%). ¹H NMR δ_{H} (CDCl₃, 300 MHz): 7.30 (m, 3H), 7.11 (d, J=0.9 Hz, 1H), 7.08 (m, 2H), 6.97 (t, J=5.4 Hz, 3H), 6.74 (d, J=5.4, 1H), 6.27 (d, J=18.9 Hz, 1H), 2.19 (dd, J=18.6, 9 Hz, 1H), 1.98 (d, J=1.5 Hz, 1H), 1.22 (s, 3H), 1.20 (s, 3H).

### Example 16

### (3-Phenoxyphenyl)methyl 3-[(Z)-2-chloro-3,3,3-trifluoro-prop-1-enyl]-2,2-dimethyl-cyclopropanecarboxylate 13

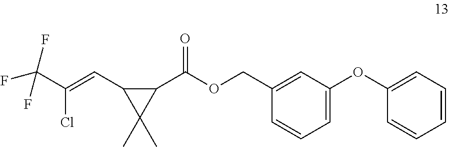 [SMILES: CC1(C)C(/C=C(\Cl)C(F)(F)F)C1C(=O)OCc1cccc(Oc2ccccc2)c1]

A solution of 3-[(Z)-2-chloro-3,3,3-trifluoro-prop-1-enyl]-2,2-dimethyl-cyclopropanecarbonyl chloride (242 mg, 1.1 eq) in toluene (6 mL) was added dropwise to a solution of (3-phenoxyphenyl)methanol (170 mg, 1 eq) and pyridine (68 μL, 1 eq) in toluene (6 mL). The reaction mixture was stirred overnight at room temperature after which time TLC analysis showed the reaction had gone to completion. The reaction mixture was diluted with ethyl acetate (15 mL) and washed with water (2×10 mL) and brine (10 mL) before being dried over MgSO₄ and the solvent removed in vacuo. The residue was purified by flash chromatography (solvent 9:1 hexane/ethyl acetate) to afford the product as a clear oil (262 mg, 73%). ¹H NMR δ_{H} (CDCl₃, 300 MHz): 7.25 (m, 4H), 6.93 (m, 6H), 5.00 (dd, J=15.6, 3.3 Hz, 2H), 2.10 (t, J=8.4 Hz, 1H), 1.95 (d, J=8.4 Hz, 1H), 1.22 (s, 3H), 1.20 (s, 3H); ESI-MS 447.1 [MNa]⁺.

### Example 17

### 3-[(Z)-2-Chloro-3,3,3-trifluoro-prop-1-enyl]-2,2-dimethyl-N-[(3-phenoxyphenyl)methyl]cyclopropanecarboxamide 55

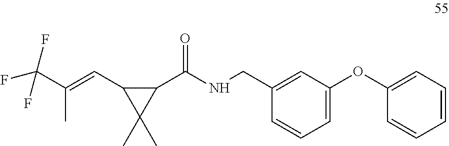 [SMILES: C/C(=C\C1C(C(=O)NCc2cccc(Oc3ccccc3)c2)C1(C)C)C(F)(F)F]

A solution of 3-[(Z)-2-chloro-3,3,3-trifluoro-prop-1-enyl]-2,2-dimethyl-cyclopropanecarbonyl chloride (100 mg, 1.1 eq) in toluene (6 mL) was added dropwise to a solution of (3-phenoxyphenyl)methanamine (170 mg, 1 eq) and pyridine (68 μL, 1 eq) in toluene (6 mL). The reaction mixture was stirred overnight at room temperature after which time TLC analysis showed the reaction had gone to completion. The reaction mixture was diluted with ethyl acetate (15 mL) and washed with water (2×10 mL) and brine (10 mL) before being dried over MgSO₄ and the solvent removed in vacuo. The residue was purified by flash chromatography (solvent 9:1 hexane/ethyl acetate) to afford the product as a clear oil (86 mg, 24%). ¹H NMR δ_{H} (CDCl₃, 300 MHz): 7.22 (m, 3H), 7.04 (m, 2H), 6.94 (m, 3H), 6.84 (m, 2H), 5.78 (s, 1H), 4.33 (ddd, J=20.7, 15.0, 5.7 Hz, 2H), 1.99 (m, 2H), 1.21 (s, 3H), 1.19 (s, 3H); ESI-MS 424.2 [MH]⁺.

### Example 18

### 3-[(Z)-2-Chloro-3,3,3-trifluoro-prop-1-enyl]-N-[cyano-(3-phenoxyphenyl)methyl]-2,2-dimethyl-cyclopropanecarboxamide 19

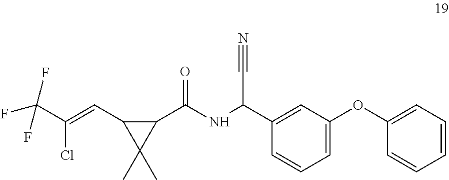 [SMILES: CC1(C)C(/C=C(\Cl)C(F)(F)F)C1C(=O)NC(C#N)c1cccc(Oc2ccccc2)c1]

A solution of 3-[(Z)-2-chloro-3,3,3-trifluoro-prop-1-enyl]-2,2-dimethyl-cyclopropanecarbonyl chloride (210 mg, 1.1 eq) in toluene (6 mL) was added dropwise to a solution of 2-amino-2-(3-phenoxyphenyl)acetonitrile (163 mg, 1 eq) and pyridine (59 μL, 1 eq) in toluene (6 mL). The reaction mixture was stirred overnight at room temperature after which time TLC analysis showed the reaction had gone to completion. The reaction mixture was diluted with ethyl acetate (15 mL) and washed with water (2×10 mL) and brine (10 mL) before being dried over MgSO₄ and the solvent removed in vacuo. The residue was purified by flash chromatography (solvent 9:1 hexane/ethyl acetate) to afford the product as a clear oil (266 mg, 73%).

¹H NMR δ_{H} (CDCl₃, 300 MHz): 7.32 (m, 3H), 7.11 (dd, J=21, 7.8 Hz, 3H), 6.82 (t, J=4.8 Hz), 6.05 (m, 2H), 2.13 (dd, J=18.3, 8.4 Hz, 1H), 1.60 (d J=8.1 Hz, 1H), 1.24 (s, 3H), 1.19 (s, 3H); ESI-MS 471.1 [MNa]⁺.

### Example 19

### (4Z)-4-Benzyloxyimino-4-[3-[(Z)-2-chloro-3,3,3-trifluoro-prop-1-enyl]-2,2-dimethyl-cyclopropyl]-2-(3-phenoxyphenyl)butanenitrile 56

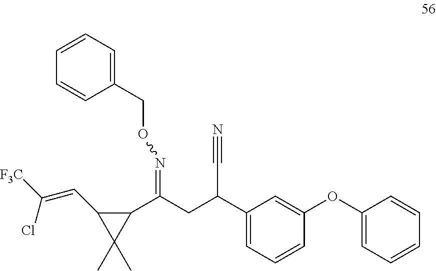 [SMILES: CC1(C)C(/C=C(\Cl)C(F)(F)F)C1C(CC(C#N)c1cccc(Oc2ccccc2)c1)=NOCc1ccccc1]

O-Benzylhydroxylamine.HCl (114 mg, 4 eq) was added to a solution of the nitrile-ketone substrate (80 mg, 1 eq) in EtOH (3 mL) and the mixture heated to 60° C. overnight, after which time the reaction was diluted with ethyl acetate (15 mL) and washed with water (2×10 mL) before being dried over MgSO₄ and the solvent removed in vacuo. The residue was purified by flash chromatography (solvent 95:5 hexane/ethyl acetate) to afford the product as a clear oil (33 mg, 33%).

¹H NMR δ_{H} (CDCl₃, 300 MHz): 7.33 (m, 8H), 7.17 (t J=5.1 Hz, 1H), 6.95 (m, 6H), 6.14 (d, J=8.7 Hz, 0.5H), 5.99 (d. J=8.7 Hz, 0.5H) 5.12 (t. J=2.7 Hz, 2H), 4.22 (m, 0.5H), 4.12 (m, 0.5H), 2.98 (m, 1H), 2.81 (m, 1H), 2.31 (m, 1H), 1.19 (d J=5.4 Hz, 3H), 1.08 (s, 3H); ESI-MS 553.2 [MH]⁺.

### Example 20

### 4-[3-[(Z)-2-Chloro-3,3,3-trifluoro-prop-1-enyl]-2,2-dimethyl-cyclopropyl]-4-oxo-2-(3-phenoxyphenyl)butanenitrile 57

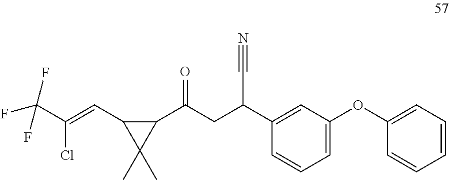 [SMILES: CC1(C)C(/C=C(\Cl)C(F)(F)F)C1C(=O)CC(C#N)c1cccc(Oc2ccccc2)c1]

To a solution of (E)-1-[3-[(Z)-2-chloro-3,3,3-trifluoro-prop-1-enyl]-2,2-dimethyl-cyclopropyl]-3-(3-phenoxyphenyl)prop-2-en-1-one (120 mg, 1 eq) in dioxane (2 mL) was added TMSCN (54 μL, 1.5 eq), Cs₂CO₃ (5 mg, 0.5 mol %) and H₂O (20 μL, 4 eq) and the mixture heated to reflux for 16 h. The reaction was quenched by the addition of 2N HCl before being extracted with ethyl acetate (3×15 mL). The organic fraction was dried over MgSO₄ and the solvent removed in vacuo. The residue was purified by flash chromatography (solvent 98:2 moving to 95:5 hexane/ethyl acetate) to afford the product as a pale yellow oil (80 mg, 63%). ¹H NMR δ_{H} (CDCl₃, 300 MHz): 7.32 (m, 3H) 7.12 (m, 2H), 7.04 (m, 3H), 6.96 (m, 2H), 6.16 (d, J=2.7 Hz, 1H), 4.34 (m, 1H), 3.28 (m, 1H), 3.08 (m, 1H), 2.62 (t, J=3.9 Hz, 1H), 2.01 (t, J=5.4 Hz, 1H), 1.28 (s, 3H), 1.25 (s, 3H); ESI-MS 470.1 [MNa]⁺.

### Example 21

### 2-[3-[(Z)-2-Chloro-3,3,3-trifluoro-prop-1-enyl]-2,2-dimethyl-cyclopropanecarbonyl]oxy-2-(3-phenoxyphenyl)acetic acid 58

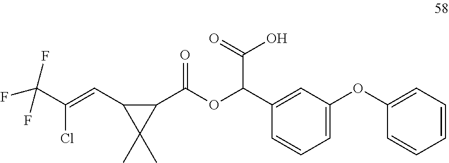 [SMILES: CC1(C)C(/C=C(\Cl)C(F)(F)F)C1C(=O)OC(C(=O)O)c1cccc(Oc2ccccc2)c1]

A solution of 3-[(Z)-2-chloro-3,3,3-trifluoro-prop-1-enyl]-2,2-dimethyl-cyclopropanecarbonyl chloride (118 mg, 0.45 mmol) in toluene (5 mL) was added dropwise to a solution of 3-phenoxymandelic acid (100 mg, 0.41 mmol) and pyridine (33 μL, 0.41 mmol) in toluene (5 mL). The reaction mixture was stirred overnight at ambient temperature after which time TLC analysis showed complete consumption of the starting material. The reaction mixture was diluted with EtOAc (15 mL) and washed with water (2×10 mL) and brine (10 mL) before being dried over MgSO₄ and the solvent removed in vacuo. The residue was purified by flash chromatography on silica gel (solvent graduated from 99.5:0.5 chloroform/acetic acid to 94.5:5:0.5 chloroform/methanol/acetic acid) to afford the product as a yellow oil.

¹H NMR δ_{H} (CDCl₃, 300 MHz): 7.30-7.24 (m, 3H), 7.19-7.04 (m, 3H), 6.96-6.90 (m, 3H), 6.80 (dd, J=8.0, 15.0 Hz, 1H), 5.81 (d, J=6.0 Hz, 1H), 2.20-2.11 (m, 1H), 2.08-2.03 (m, 1H), 1.27-1.20 (m, 6H). ESI-MS 492.3 [MNa]⁺.

### Example 22

### 2-[[3-[(Z)-2-Chloro-3,3,3-trifluoro-prop-1-enyl]-2,2-dimethyl-cyclopropyl]methylamino]-2-(3-phenoxyphenyl)acetonitrile 20

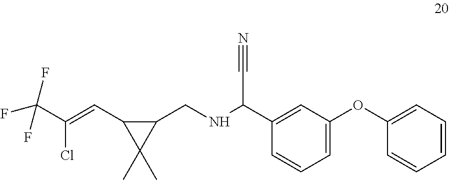 [SMILES: CC1(C)C(/C=C(\Cl)C(F)(F)F)C1CNC(C#N)c1cccc(Oc2ccccc2)c1]

Sodium triacetoxyborohydride (142 mg, 0.67 mmol) was added to a solution of 3-[(Z)-2-chloro-3,3,3-trifluoro-prop-1-enyl]-2,2-dimethyl-cyclopropanecarbaldehyde (101 mg, 0.45 mmol) and 2-amino-2-(3-phenoxyphenyl)acetonitrile (100 mg, 0.45 mmol) in DCE (2 mL) in the presence of molecular sieves. The reaction mixture was stirred overnight at ambient temperature after which time TLC analysis showed complete consumption of the starting material. The reaction mixture was diluted with EtOAc (15 mL) and washed with water (2×10 mL) and brine (10 mL) before being dried over MgSO₄ and the solvent removed in vacuo. The residue was purified by flash chromatography on silica gel (solvent 90:10 hexane/EtOAc) to afford the product as a colourless oil (67 mg, 35%).

¹H NMR δ_{H} (CDCl₃, 300 MHz): 7.29 (t, J=8.0 Hz, 3H), 7.19-7.04 (m, 3H), 6.96 (t, J=8.0 Hz, 3H), 6.12 (td, J=1.5, 11.0 Hz, 1H), 4.70 (d, J=11.5 Hz, 1H), 2.89-2.79 (m, 1H), 2.72-2.63 (m, 1H), 1.70-1.63 (m, 1H), 1.50 (s, 2H), 1.13 (d, J=3.5 Hz, 3H), 1.05 (d, J=3.5 Hz, 3H). ESI-MS 435.1 [MH]⁺.

### Example 23

### (E)-1-[3-[(Z)-2-Chloro-3,3,3-trifluoro-prop-1-enyl]-2,2-dimethyl-cyclopropyl]-3-(3-phenoxyphenyl)prop-2-en-1-one 59

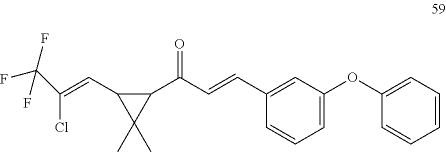 [SMILES: CC1(C)C(/C=C(\Cl)C(F)(F)F)C1C(=O)/C=C/c1cccc(Oc2ccccc2)c1]

To an ice-cooled solution of 1-[3-[(Z)-2-chloro-3,3,3-trifluoro-prop-1-enyl]-2,2-dimethyl-cyclopropyl]ethanone (100 mg, 0.42 mmol) in EtOH (1 mL) was added 10% NaOH solution (1 mL) followed by 3-phenoxybenzaldehyde (72 μL, 0.42 mmol). The reaction mixture was stirred overnight at ambient temperature after which time TLC analysis showed complete consumption of the starting material. The reaction mixture was extracted with EtOAc (3×2.5 mL) and washed with H₂O (5 mL) before being dried over MgSO₄ and the solvent removed in vacuo. The residue was purified by flash chromatography on silica gel (solvent 95:5 hexane/EtOAc) to afford the product as an oil (130 mg, 74%).

¹H NMR δ_{H} (CDCl₃, 300 MHz): 7.41 (d, J=16.0 Hz, 1H), 7.30 (t, J=6.5 Hz, 3H), 7.23-7.19 (m, 1H), 7.13-7.03 (m, 2H), 7.00-6.93 (m, 3H), 6.73 (d, J=16.0 Hz, 1H), 6.16 (d, J=10.0 Hz, 1H), 2.64-2.59 (m, 1H), 2.25 (d, J=5.0 Hz, 1H), 1.27 (s, 3H), 1.18 (s, 3H). ESI-MS 422.9 [MH]⁺.

### Example 24

### [2-Ethoxy-2-oxo-1-(3-phenoxyphenyl)ethyl] 3-[(Z)-2-chloro-3,3,3-trifluoro-prop-1-enyl]-2,2-dimethyl-cyclopropanecarboxylate 60

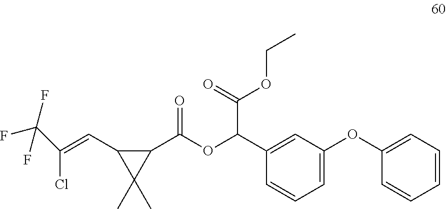 [SMILES: CCOC(=O)C(OC(=O)C1C(/C=C(\Cl)C(F)(F)F)C1(C)C)c1cccc(Oc2ccccc2)c1]

A solution of 3-[(Z)-2-chloro-3,3,3-trifluoro-prop-1-enyl]-2,2-dimethyl-cyclopropanecarbonyl chloride (278 mg, 1.06 mmol) in toluene (6 mL) was added dropwise to a solution of ethyl 2-hydroxy-2-(3-phenoxyphenyl)acetate (223 mg, 0.82 mmol) and pyridine (90 μL, 1.06 mmol) in toluene (5 mL). The reaction mixture was stirred overnight at ambient temperature after which time TLC analysis showed complete consumption of the starting material. The reaction mixture was diluted with EtOAc (15 mL) and washed with water (2×10 mL) and brine (10 mL) before being dried over MgSO₄ and the solvent removed in vacuo. The residue was purified by flash chromatography on silica gel (solvent 98:2 hexane/EtOAc) to afford the product as a white solid (252 mg, 62%).

¹H NMR δ_{H} (CDCl₃, 300 MHz): 7.30-7.24 (m, 3H), 7.14-7.03 (m, 3H), 6.95-6.90 (m, 3H), 6.80 (dd, J=9.0, 14.5 Hz, 1H), 5.78 (d, J=2.5 Hz, 1H), 4.16-4.02 (m, 2H), 2.20-2.05 (m, 2H), 1.27-1.10 (m, 9H). ESI-MS 519.1 [MNa]⁺.

### Example 25

### 1-(4,6-Dimethoxypyrimidin-2-yl)-3-[2-(hydroxymethyl)-5-(methanesulfonamidomethyl)phenyl]sulfonyl-urea 8

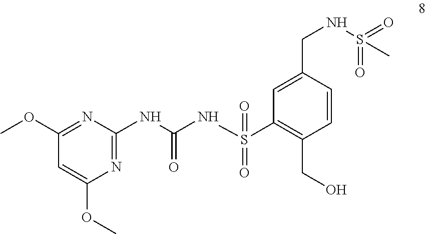

To a solution of methyl 2-[(4,6-dimethoxypyrimidin-2-yl)carbamoylsulfamoyl]-4-(methanesulfonamidomethyl)benzoate (2 g, 3.98 mmol) in THF (30 mL) was added lithium aluminium hydride (755 mg, 19.88 mmol), portionwise, at −20° C. The reaction mixture was warmed to ambient temperature over 1 h, after which time TLC showed complete consumption of the starting material. The reaction was quenched with IPA (5 mL), MeOH (5 mL) and H₂O and then acidified to pH 3 with 2 M HCl before being extracted with EtOAc, the organic layer dried over MgSO₄ and the solvent removed in vacuo. The resulting product was purified by flash chromatography on silica gel (solvent EtOAc) to afford the desired product as a white solid (1.12 g, 59%).

¹H NMR δ_{H} (CDCl₃, 300 MHz): 9.11 (s, 1H), 7.90 (s, 1H), 7.53-7.51 (m, 1H), 7.40-7.38 (m, 1H), 7.24-7.20 (m, 1H), 5.51 (s, 1H), 4.75 (s, 2H), 4.06 (d, J=6.0 Hz, 2H), 3.70 (s, 6H), 2.60 (br, 2H), 2.56, (s 3H). ESI-MS 476.1 [MH]⁺.

### Example 26

### [2-[(4,6-Dimethoxypyrimidin-2-yl)carbamoylsulfamoyl]-4-(methanesulfonamidomethyl)phenyl]methylacetate 61

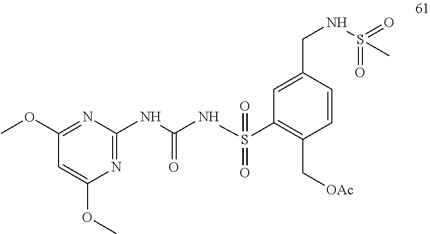

Acetic anhydride (0.12 mL, 1.28 mmol) was added to a solution of 1-(4,6-dimethoxypyrimidin-2-yl)-3-[2-(hydroxymethyl)-5-(methanesulfonamidomethyl)phenyl]sulfonyl-urea (200 mg, 0.42 mmol) and triethylamine (0.18 mL, 1.28 mmol) in DCM (3 mL). The reaction mixture was stirred at ambient temperature for 23 h, after which time TLC showed complete consumption of the starting material. The reaction was diluted with EtOAc (20 mL) and washed with H₂O (20 mL) before being dried over MgSO₄ and the solvent removed in vacuo. The crude material was purified by flash chromatography on silica gel (solvent EtOAc) to afford the product as a white solid (96 mg, 44%).

¹H NMR δ_{H} (CDCl₃, 300 MHz): 8.21 (s, 1H), 7.65-7.59 (m, 2H), 7.55 (d, J=8.0 Hz, 1H), 5.73 (s, 1H), 5.45 (s, 2H), 4.36 (d, J=6.5 Hz, 2H), 3.90 (s, 6H), 2.87 (s, 3H), 1.93 (s, 3H). ESI-MS 518.1 [MH]⁺.

### Example 27

### Ethyl 2-[4-[[5-(trifluoromethyl)-2-pyridyl]oxy]phenoxy]propanoate 62

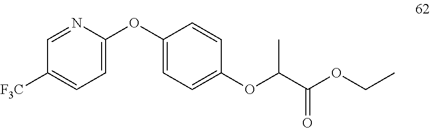 [SMILES: CCOC(=O)C(C)Oc1ccc(Oc2ccc(C(F)(F)F)cn2)cc1]

A suspension of 4-[[5-(trifluoromethyl)-2-pyridyl]oxy]phenol (6 g, 23.51 mmol), ethyl 2-bromopropanoate (3.05 mL, 23.51 mmol) and potassium carbonate (3.57 g, 25.86 mmol) in acetonitrile (60 mL) was heated at 70° C. for 16 h, after which time TLC showed complete consumption of the starting material. The reaction mixture was filtered and the resulting filtrate was dried in vacuo. The crude material was purified by flash chromatography on silica gel (solvent 90:10 hexane/EtOAc) to afford the product as a colourless oil (6.91 g, 83%).

¹H NMR δ_{H} (CDCl₃, 300 MHz): 8.36 (s, 1H), 7.80 (dd, J=2.5, 8.5 Hz, 1H), 7.01-6.96 (m, 2H), 6.88-6.83 (m, 3H), 4.66 (q, J=7.0 Hz, 1H), 4.17 (q, J=7.0 Hz, 2H), 1.54 (d, J=7.0 Hz, 3H), 1.20 (t, J=7.0 Hz, 3H). ESI-MS 356.0 [MH]⁺.

### Example 28

### 2-[4-[[5-(Trifluoromethyl)-2-pyridyl]oxy]phenoxy]propan-1-ol 27

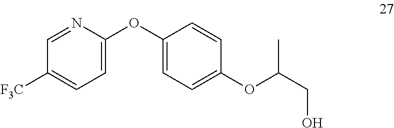 [SMILES: CC(CO)Oc1ccc(Oc2ccc(C(F)(F)F)cn2)cc1]

A solution of ethyl 2-[4-[[5-(trifluoromethyl)-2-pyridyl]oxy]phenoxy]propanoate (1 g, 5.63 mmol) in THF (25 mL) was added dropwise to an ice-cooled suspension of lithium aluminium hydride (257 mg, 6.75 mmol) in THF (25 mL). The reaction mixture was warmed to ambient temperature overnight after which time TLC analysis showed complete consumption of the starting material. The reaction was then cooled to 0° C. and quenched with H₂O and extracted with EtOAc before being dried over MgSO₄ and the solvent removed in vacuo. The crude material was purified by flash chromatography on silica gel (solvent 98:2 DCM:MeOH) to afford the product as a yellow oil (1.6 g, 91%).

¹H NMR δ_{H} (CDCl₃, 300 MHz): 8.37 (s, 1H), 7.81 (dd, J=2.5, 8.5 Hz, 1H), 7.02-6.97 (m, 2H), 6.89-6.85 (m, 3H), 4.46-4.36 (m, 1H), 3.67-3.62 (m, 2H), 1.22 (d, J=6.0 Hz, 3H). ESI-MS 314.0 [MH]⁺.

### Example 29

### 2-[4-[[5-(Trifluoromethyl)-2-pyridyl]oxy]phenoxy]propanal 28

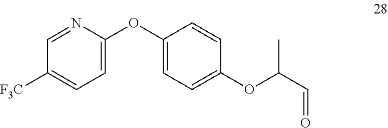 [SMILES: CC(C=O)Oc1ccc(Oc2ccc(C(F)(F)F)cn2)cc1]

To a solution of Dess-Martin periodinane (1.40 g, 3.29 mmol) in DCM (30 mL) was added a solution of 2-[4-[[5-(trifluoromethyl)-2-pyridyl]oxy]phenoxy]propan-1-ol (860 mg, 2.75 mmol) in DCM (30 mL) over a period of 15 mins. The reaction mixture was stirred at ambient temperature for 2 h, after which time TLC showed complete consumption of the starting material. The solvent was removed in vacuo, then Et₂O (150 mL) was added and the resulting suspension was filtered and the resulting filtrate was dried in vacuo. The crude material was purified by flash chromatography on silica gel (solvent graduated from DCM to 95:5 DCM:MeOH) to afford the product as a pale yellow oil (650 mg, 76%).

¹H NMR δ_{H} (CDCl₃, 300 MHz): 9.77 (s, 1H), 8.45 (s, 1H), 7.91 (dd, J=2.5, 8.5 Hz, 1H), 7.13-7.07 (m, 2H), 7.00-6.81 (m, 3H), 4.68-4.61 (m, 1H), 1.56 (d, J=6.0 Hz, 3H). ESI-MS 312.0 [MH]⁺.

### Example 30

### 2-[4-[[5-(Trifluoromethyl)-2-pyridyl]oxy]phenoxy]propyl acetate 63

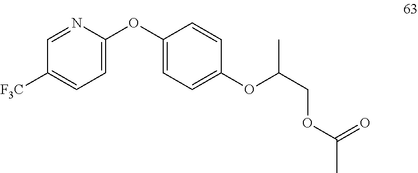 [SMILES: CC(=O)OCC(C)Oc1ccc(Oc2ccc(C(F)(F)F)cn2)cc1]

Acetic anhydride (0.12 mL, 1.28 mmol) was added to an ice-cooled solution of 2-[4-[[5-(trifluoromethyl)-2-pyridyl]oxy]phenoxy]propan-1-ol (200 mg, 0.64 mmol) and triethylamine (0.18 mL, 1.28 mmol) in DCM (3 mL). The reaction mixture was warmed at ambient temperature over 16 h, after which time TLC showed complete consumption of the starting material. The reaction was diluted with DCM (20 mL) and washed with H₂O (20 mL) before being dried over MgSO₄ and the solvent removed in vacuo. The crude material was purified by flash chromatography on silica gel (solvent 80:20 Hexane:EtOAc) to afford the product as a colourless oil (193 mg, 85%).

¹H NMR δ_{H} (CDCl₃, 300 MHz): 8.46 (s, 1H), 7.90 (dd, J=2.5, 8.5 Hz, 1H), 7.11-7.06 (m, 2H), 7.00-6.97 (m, 3H), 4.66-4.56 (m, 1H), 4.33-4.16 (m, 2H), 2.10 (s, 3H), 1.37 (d, J=6.0 Hz, 3H). ESI-MS 356.1 [MH]⁺.

### Example 31

### (1E)-2-[4-[[5-(Trifluoromethyl)-2-pyridyl]oxy]phenoxy]propanal oxime 64

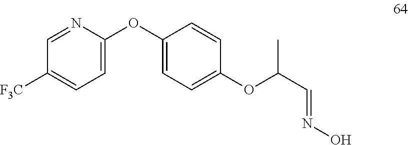 [SMILES: CC(/C=N/O)Oc1ccc(Oc2ccc(C(F)(F)F)cn2)cc1]

Hydroxylamine hydrochloride (246 mg, 3.53 mmol) was added to a suspension of 2-[4-[[5-(trifluoromethyl)-2-pyridyl]oxy]phenoxy]propanal (275 mg, 0.88 mmol) and sodium carbonate (375 mg, 3.53 mmol) in EtOH (9 mL). The reaction mixture was heated at 70° C. overnight, after which time LCMS showed complete consumption of the starting material. The reaction mixture was diluted with EtOAc, then washed with water and brine before being dried over MgSO₄ and the solvent removed in vacuo. The crude material was purified by flash chromatography on silica gel (solvent 80:20 hexane/EtOAc) followed by recrystallisation (solvent 99:1 hexane:EtOAc) to afford the product as a white solid (67 mg, 23%).

¹H NMR δ_{H} (CDCl₃, 300 MHz): 8.46 (s, 1H), 7.89 (dd, J=2.5, 8.5 Hz, 1H), 7.74 (br, 0.6H), 7.44 (d, J=7.0 Hz, 0.6H), 7.36 (br, 0.4H), 7.09-6.90 (m, 5H), (d, J=6.0 Hz, 0.4H), 5.56-5.47 (m, 0.4H), 4.99-4.90 (m, 0.6H), 1.56 (d, J=6.5 Hz, 3H). ESI-MS 327.0 [MH]⁺.

### Example 32

### N-Benzyloxy-2-[4-[[5-(trifluoromethyl)-2-pyridyl]oxy]phenoxy]propan-1-imine 65

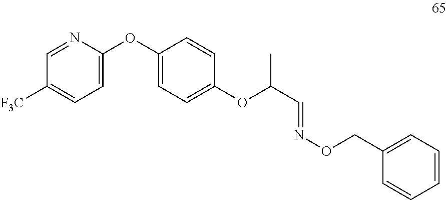 [SMILES: CC(/C=N/OCc1ccccc1)Oc1ccc(Oc2ccc(C(F)(F)F)cn2)cc1]

o-Benzylhydroxylamine hydrochloride (564 mg, 3.53 mmol) was added to a suspension of 2-[4-[[5-(trifluoromethyl)-2-pyridyl]oxy]phenoxy]propanal (275 mg, 0.88 mmol) and sodium carbonate (375 mg, 3.53 mmol) in EtOH (9 mL). The reaction mixture was heated at 70° C. for 16 h, after which time TLC showed complete consumption of the starting material. The reaction mixture was diluted with EtOAc, then washed with water and brine before being dried over MgSO₄ and the solvent removed in vacuo. The crude material was purified by flash chromatography on silica gel (solvent graduated from 98:2 hexane/EtOAc to 93:7 hexane/EtOAc) to afford the product as a white solid (212 mg, 58%).

¹H NMR δ_{H} (CDCl₃, 300 MHz): 8.46 (s, 1H), 7.90 (dd, J=2.5, 8.5 Hz, 1H), 7.44-7.29 (m, 6H), 7.05-6.94 (m, 4H), 6.84 (d, J=9.5 Hz, 0.7H), 6.79 (d, J=6.0 Hz, 0.3H), 5.47-5.39 (m, 0.3H), 5.19 (s, 0.7H), 5.12 (s, 1.4H), 4.97-4.88 (m, 0.7H), 1.54 (d, J=6.5 Hz, 3H). ESI-MS 417.1 [MH]⁺.

### Example 33

### sec-Butyl 2-(2-oxoethyl)piperidine-1-carboxylate 39

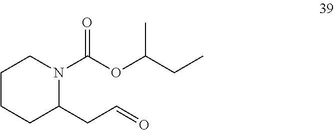 [SMILES: CCC(C)OC(=O)N1CCCCC1CC=O]

To a solution of Dess-Martin periodinane (1.11 g, 2.52 mmol) in DCM (25 mL) was added a solution of Icaridin (500 mg, 2.18 mmol) in DCM (25 mL) under nitrogen. The reaction mixture was stirred at ambient temperature for 20 h, after which time TLC showed complete consumption of the starting material. The solvent was removed in vacuo and the crude material was purified by flash chromatography on silica gel (solvent graduated from DCM to 95:5 DCM:MeOH). Hexane was added to the resulting oil the suspension formed was filtered and the resulting filtrate was dried in vacuo to afford the product as a colourless oil (309 mg, 62%).

NMR δ_{H} (CDCl₃, 300 MHz): 9.75 (s, 1H), 4.91 (br, 1H), 4.82-4.72 (m, 1H), 4.09-4.04 (m, 1H), 2.88-2.71 (m, 1H), 2.65-2.54 (m, 1H), 1.77-1.41 (m, 9H), 1.23-1.18 (m, 3H), 0.93-0.91 (m, 3H). ESI-MS 477.2 [M₂Na]⁺ or EI-MS 227.2 [M].

### Example 34

### sec-Butyl 2-(2-hydroxyimino)ethyl)piperidine-1-carboxylate 40a

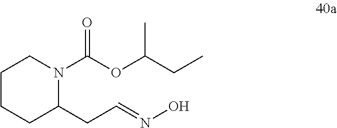 [SMILES: CCC(C)OC(=O)N1CCCCC1C/C=N/O]

Hydroxylamine hydrochloride (196 mg, 2.82 mmol) was added to a suspension of sec-butyl 2-(2-oxoethyl)piperidine-1-carboxylate (160 mg, 0.70 mmol) and sodium carbonate (298 mg, 2.81 mmol) in MeOH (5 mL). The reaction mixture was heated at reflux for 18.5 h, after which time TLC showed complete consumption of the starting material and the solvent was removed in vacuo. H₂O (25 mL) was added then the mixture was extracted with EtOAc (3×20 mL) and washed with brine (2×20 mL) before being dried over MgSO₄ and the solvent removed in vacuo. The crude material was purified by flash chromatography on silica gel (solvent 60:40 hexane/EtOAc) to afford the product as a colourless oil (141 mg, 83%). as a mixture of both E and Z isomers.

NMR δ_{H} (CDCl₃, 300 MHz): 8.14, 7.66, 7.39-7.36, 6.75 (4 signals, 2H), 4.78-7.72 (m, 1H), 4.52 (br, 1H), 4.06-4.02 (m, 1H), 2.91-2.78 (m, 1H), 2.67-2.49 (m, 1H), 2.40-2.29 (m, 1H), 1.65-1.40 (m, 8H), 1.22-1.18 (m, 3H), 0.93-0.91 (m, 3H). ESI-MS 507.33 [M₂Na]⁺.

### Example 34

### 2-(1-sec-Butoxycarbonyl-2-piperidyl)acetic acid 41

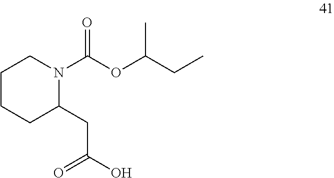 [SMILES: CCC(C)OC(=O)N1CCCCC1CC(=O)O]

Concentrated sulfuric acid (0.49 mL) was added dropwise to a solution of sodium dichromate (650 mg, 2.18 mmol) in water (3 mL) and the solution was then added dropwise to an ice-cooled solution of Icaridin (500 mg, 2.18 mmol) in acetone (30 mL). The reaction mixture was heated at 40° C. for 16 hours, after which time TLC analysis showed complete consumption of the starting material. The reaction mixture was diluted with water (20 mL) then filtered and the acetone removed in vacuo. The aqueous phase was extracted with EtOAc (3×25 mL) and washed with brine (2×50 mL) before being dried over MgSO₄ and the solvent removed in vacuo. The resulting solid was washed successively with EtOAc, hexanes then DCM and the solvent removed in vacuo. Water (15 mL) was added to the residue and extracted with DCM (3×10 mL) before being dried over MgSO₄ and the solvent removed in vacuo to afford the product as a colourless oil (487 mg, 92%).

NMR δ_{H} (CDCl₃, 300 MHz): 4.73-4.66 (m, 2H), 4.00-3.95 (m, 1H), 2.80-2.71 (m, 1H), 2.63-2.47 (m, 2H), 1.60-1.33 (m, 8H), 1.15-1.11 (m, 3H), 0.85-0.81 (m, 3H). ESI-MS 537.3-[M₂Na]⁺.

### Example 36

### sec-Butyl 2-(2-methoxy-2-oxo-ethyl)piperidine-1-carboxylate 42a

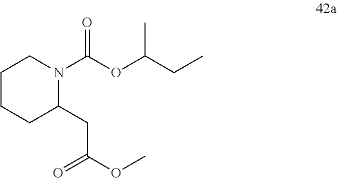 [SMILES: CCC(C)OC(=O)N1CCCCC1CC(=O)OC]

Sulfuric acid (5 drops from a Pasteur pipette) was added to a solution of 2-(1-sec-butoxycarbonyl-2-piperidyl)acetic acid (407 mg, 1.67 mmol) in methanol (10 mL). The reaction mixture was heated at reflux for 18 h, after which time TLC showed complete consumption of the starting material and the solvent was removed in vacuo. H₂O (15 mL) was added and the mixture was extracted with EtOAc (3×15 mL) before being dried over MgSO₄ and the solvent removed in vacuo to afford the product as a colourless oil (397 mg, 92%). NMR δ_{H} (CDCl₃, 300 MHz): 4.71-4.64 (m, 2H), 3.99-3.95 (m, 1H), 3.59 (s, 3H), 2.79-2.71 (m, 1H), 2.58-2.44 (m, 2H), 1.59-1.32 (m, 8H), 1.15-1.11 (m, 3H), 0.85-0.81 (m, 3H). EI-MS 258.3 [MH]⁺.

### Example 37

### 2-[[5-Bromo-2-(3-chloro-2-pyridyl)pyrazole-3-carbonyl]amino]-5-cyano-3-methyl-benzoic acid 66

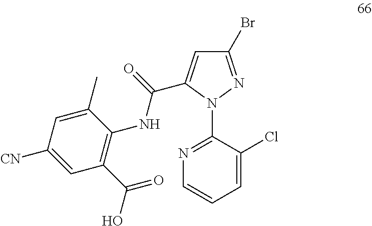

A solution of 2-amino-5-cyano-3-methyl-benzoic acid (135 mg, 0.77 mmol) in THF (5 mL) was added dropwise to a solution of 5-bromo-2-(3-chloro-2-pyridyl)pyrazole-3-carbonyl chloride (271 mg, 0.84 mmol) in THF (5 mL) under nitrogen. Triethylamine (0.12 mL, 0.84 mmol) was then added and the reaction mixture was stirred at ambient temperature for 18 h, after which time TLC showed complete consumption of the starting material. The reaction mixture was diluted with water (10 mL) and extracted with EtOAc (3×10 mL) before being dried over MgSO₄ and the solvent removed in vacuo. The residue was purified by flash chromatography on silica gel (solvent 70:30 hexane:EtOAc) to afford the desired product as a yellow solid (80 mg, 23%).

NMR δ_{H} (CDCl₃, 300 MHz): 8.49 (d, J=5.0 Hz, 1H), 8.24 (s, 1H), 7.91 (d, J=8.0 Hz, 1H), 7.67 (s, 1H), 7.47-7.43 (m, 1H), 7.25 (s, 1H), 1.79 (s, 3H). ESI-MS 459.9 [M-H]−.

### Example 38

### Methyl 2-[[5-bromo-2-(3-chloro-2-pyridyl)pyrazole-3-carbonyl]amino]-5-cyano-3-methyl-benzoate 67

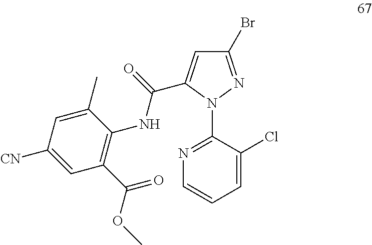

Thionyl chloride (0.10 mL, 1.55 mmol) was added to a solution of 2-[[5-bromo-2-(3-chloro-2-pyridyl)pyrazole-3-carbonyl]amino]-5-cyano-3-methyl-benzoic acid (210 mg, 0.46 mmol) in toluene (5 mL) under nitrogen. The reaction mixture was heated at reflux for 18 h, after which time TLC showed complete consumption of the starting material. The volatiles were removed in vacuo before methanol (5 mL) and triethylamine (0.6 mL, 0.46 mmol) were added and the reaction mixture was heated at reflux for 4 h. After cooling to ambient temperature, the reaction was diluted with water (10 mL) and extracted with EtOAc (3×10 mL), before being dried over MgSO₄ and the solvent removed in vacuo. The crude material was purified by flash chromatography on silica gel (solvent 60:40 hexane:EtOAc) to afford the product as a white solid (107 mg, 49%).

NMR δ_{H} (CDCl₃, 300 MHz): 10.41 (s, 1H), 8.38 (d, J=4.5 Hz, 1H), 8.06 (s, 1H), 7.82 (d, J=4.5 Hz, 1H), 7.85 (s, 1H), 7.34-7.32 (m, 1H), 7.00 (s, 1H), 3.88 (s, 3H), 1.96 (s, 3H). ESI-MS 476.1 [MH]⁺.

### Example 39

### 2-[[5-bromo-2-(3-chloro-2-pyridyl)pyrazol-3-yl]methyleneamino]-5-cyano-N,3-dimethylbenzamide 54

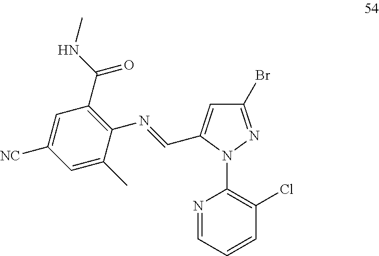 [SMILES: CNC(=O)c1cc(C#N)cc(C)c1/N=C/c1cc(Br)nn1-c1ncccc1Cl]

A solution of 3-bromo-1-(3-chloro-2-pyridinyl)-1H-pyrazole-5-carboxaldehyde (110 mg, 0.38 mmol) and 2-amino-5-cyano-N,3-dimethylbenzamide (73 mg, 0.38 mmol) in toluene (5 mL) was heated to reflux and water was continuously removed using a Dean-Stark apparatus. After 7 days the mixture was allowed to cool to room temperature. Ethyl acetate (20 mL) was added and the mixture was filtered and evaporated under reduced pressure to give the product as a white solid (130 mg, 75%).

¹H NMR δ_{H} (CDCl₃, 300 MHz): 8.42 (d, J=4.5 Hz, 1H), 8.07 (s, 1H), 8.00 (d, J=8.0 Hz, 1H), 7.40 (dd, J=8.0, 3.5 Hz, 1H), 7.29 (s, 1H), 6.62 (s, 1H), 6.20 (s, 1H), 5.71 (d, J=2 Hz, 1H), 3.00 (s, 3H), 2.07 (s, 3H). ESI-MS 459.1 [MH⁺].

### Example 40

### Testing the Insecticidal Activity of Cyhalothrin and Cyantraniliprole Analogues

A laboratory bioassay was conducted to screen 14 compounds (cyhalothrin (15); cyantraniliprole; 9 cyhalothrin analogues: 13, 55, 19, 57, 56, 58, 20, 59 and 60; and 3 cyantraniliprole analogues: 66, 67, and 54) for biocidal activity against aphids, Myzus persicae, mosquito larvae, Aedes aegypti, cabbage moth larvae, Mamestra brassicae, and two-spotted spider mites, Tetranychus urticae, in terms of knockdown and mortality. Compounds were diluted in DMSO and assessed at a range of concentrations from 0.5% to 0.00001%. A DMSO only negative control was also included for comparative purposes. These were applied directly onto the insects/mites and assessments of knockdown and mortality were conducted at 24 and 48 hours post treatment.

### Test System

Aphids, Myzus persicae, were originally obtained from a laboratory culture maintained at the Food and Environment Research agency (York, UK) and maintained on Chinese cabbage plants at i2LResearch. Mixed sex and age aphids were used in the experiments.

Mosquitoes, Aedes aegypti, were obtained as eggs from a laboratory culture maintained at The London School of Hygiene and Tropical Medicine (London, UK) and reared to 3^{rd} instar larvae at i2LResearch, prior to use in the experiments.

Cabbage moths, Mamestra brassicae, were obtained as eggs from a laboratory culture maintained from the Centre for Ecology and Hydrology (Oxfordshire, UK) and reared on Chinese cabbage plants to 2^{nd} instar larvae, prior to use in the experiments.

Two-spotted spider mites, Tetranychus urticae, were obtained from a standard susceptible laboratory culture maintained at Syngenta Bioline (Essex, UK). Mixed sex and age mites were used in the experiments.

The temperature was maintained between 22.1° C. and 24.8° C. and the relative humidity ranged from 26.1% to 44.2%. Arthropods were maintained on a 16:8 hour (light:dark) photoperiod post treatment.

### Test Treatments and Application

The test compounds were dissolved in DMSO (Dimethyl sulfoxide) and diluted at a range of six concentrations: 0.5%, 0.1%, 0.01%, 0.001%, 0.0001% and 0.00001%. In the field, 0.05% represents the normal dosage applied. Activity at this level or at dilutions less than this is thus indicative of an effective compound. For the mites and caterpillars in some cases the 0.5% was not conducted due to the limited amount of compound available. The concentrates were prepared at room temperature and stirred for approximately 15 minutes, using a vortex mixer. A negative control (DMSO only) was also included in the testing for comparative purposes. Treatments were applied directly onto the arthropods within Petri dishes, using a Potter tower, at a rate of 0.2 ml per replicate.

### Experimental Design

Approximately twenty aphids/mites and ten moth larvae were counted into a 55 mm diameter Petri dish lined with a leaf disc (abaxial surface upwards) mounted on damp cotton wool. For moth larvae and aphids, leaf discs were cut from round cabbage, for mites, leaf discs were cut from dwarf French bean plants. Twenty mosquito larvae were placed into an 11 cm diameter plastic container, filled with approximately 150 ml of de-chlorinated tap water, using a pipette.

The aphids, mites and moth larvae were sprayed using the Potter tower. The mosquitoes were sprayed using a Gilson pipette. The number of knocked down and dead arthropods was assessed at 24 and 48 hours post treatment.

Three to five replicates were performed for each treatment, for each species.

### Results

The results are shown in Tables 1 and 2. If a compound showed over 80% control over the target species at that concentration it was assigned an A, if it showed 50%-80% control it was assigned a B and if it showed less than 50% control, it was assigned a C.

TABLE 1
Cyantraniliprole analogues

COMPOUND

| SPECIES | CONC. | Cyantraniliprole | 66 | 67 | 54 |
|---|---|---|---|---|---|
| Mosquito | 0.0000001 | C | C | C | C |
| Larvae | 0.000001 | C | C | C | C |
| | 0.00001 | C | C | C | C |
| | 0.0001 | C | C | C | C |
| | 0.001 | C | C | C | C |
| | 0.005 | B | B | C | B |
| Cabbage | 0.0000001 | C | C | C | C |
| Moth | 0.000001 | C | C | C | C |
| Caterpillar | 0.00001 | B | C | C | C |
| | 0.0001 | A | C | C | C |
| | 0.001 | A | C | A | B |
| | 0.005 | A | B | A | B |
| Aphids | 0.0000001 | C | C | C | B |
| | 0.000001 | C | C | C | B |
| | 0.00001 | C | C | C | B |
| | 0.0001 | C | C | C | A |
| | 0.001 | B | C | C | A |
| | 0.005 | A | C | C | A |
| Spider | 0.0000001 | C | C | C | C |
| Mites | 0.000001 | C | C | C | C |
| | 0.00001 | C | C | C | B |
| | 0.0001 | C | C | C | A |
| | 0.001 | C | C | C | A |
| | 0.005 | B | C | C | A |

TABLE 2
Cyhalothrin analogues

| | | COMPOUND | | | | | | | | | |
|---|---|---|---|---|---|---|---|---|---|---|---|
| SPECIES | CONC. | Cyhalothrin (15) | 13 | 55 | 19 | 57 | 56 | 58 | 20 | 59 | 60 |
| Mosquito | 0.0000001 | C | C | C | C | B | C | C | C | C | C |
| Larvae | 0.000001 | A | C | C | C | A | C | C | C | C | C |
| | 0.00001 | A | C | C | C | A | C | C | C | C | C |
| | 0.0001 | A | C | C | A | A | C | C | C | C | A |
| | 0.001 | A | B | A | A | A | C | A | B | A | A |
| | 0.005 | A | A | A | A | A | C | A | A | A | A |
| Cabbage | 0.0000001 | C | A | A | C | C | C | C | C | C | C |
| Moth | 0.000001 | B | A | A | B | C | C | C | C | C | C |
| Caterpillar | 0.00001 | A | A | A | B | C | C | C | C | C | C |
| | 0.0001 | A | A | A | A | A | C | B | C | C | A |
| | 0.001 | A | A | A | A | A | B | A | C | A | A |
| | 0.005 | A | A | A | A | A | — | A | A | A | A |
| Aphids | 0.0000001 | C | C | C | C | C | C | B | C | C | C |
| | 0.000001 | C | B | C | C | C | C | B | C | C | C |
| | 0.00001 | C | A | C | B | C | C | A | C | C | A |
| | 0.0001 | C | A | C | A | A | C | A | C | B | A |
| | 0.001 | A | A | C | A | A | B | A | C | B | A |
| | 0.005 | A | A | A | A | A | A | A | A | A | A |
| Spider Mites | 0.0000001 | C | C | B | C | C | C | C | C | C | C |
| | 0.000001 | B | B | B | C | C | C | B | C | B | B |
| | 0.00001 | A | A | A | B | B | C | B | C | A | B |
| | 0.0001 | A | A | A | A | A | C | B | C | B | A |
| | 0.001 | A | A | A | A | A | B | B | C | A | A |
| | 0.005 | A | A | A | A | A | — | A | B | A | A |

### Example 41

### Testing the Insect Repellent Activity of Icaridin Analogues

A laboratory bioassay was conducted to screen 5 compounds (Icaridin (38); and 4 icaridin analogues: 39, 40a, 42a and 41) for repellent activity against houseflies, Musca domestica, black ants, Lasius niger, German cockroaches, Blattella germanica, and bedbugs, Cimex lectularius. Compounds were diluted in a mixture of ethanol and water and assessed at a 20% concentration. A mixture of ethanol/water only negative control was also included for comparative purposes. These were applied onto an aluminium foil tile, which was placed into one half of an arena. The other half of the arena contained an untreated aluminium foil tile. The number of insects present in the treated and untreated area was assessed every 5 minutes for a total of 20 minutes.

### Test System

Houseflies, Musca domestica, and German cockroaches, Blattella germanica, were obtained from a laboratory culture maintained at i2LResearch. Mixed sex and age adult insects (flies were aged 3-6 days old) were used in the experiments.

Black ants, Lasius niger, were field collected from the Cardiff area. Mixed age worker ants were used in the experiments.

Bedbugs, Cimex lectularius, were obtained from a laboratory culture maintained at CimexStore (Chepstow, UK). Mixed age and sex adult bedbugs were used in the experiments (see deviation below).

Temperature was maintained between 24.2 and 24.6° C. and relative humidity ranged between 27.5% and 43.5%, throughout the experimental period.

### Test Treatments and Application

The tested compounds were diluted in ethanol and water to a concentration of 20% (w/w: compound 20%, ethanol 40%, water 40%). The concentrates were prepared at room temperature and stirred for approximately 15 minutes using a vortex mixer. A negative control (mixture of ethanol:water 50:50) was also included in the testing for comparative purposes. Treatments were applied directly on to a non porous surface (aluminium foil), using a Gilson pipette at a rate of 0.225 ml per 225 cm² surface tile. A small piece of acetate was used to evenly spread the treatments across the entire surface of the tile.

### Experimental Design

For the testing against houseflies: A clear plastic container was used measuring approximately 34 cm long×21 cm wide×20 cm high, with a piece of netting and lid on top. This was divided into two halves using an additional tile with a small slit measuring approximately 2.5 cm×5 cm cut at a height of approximately 20 cm from the base, so that the insects could freely travel between halves. A treated and untreated tile was placed either side of the dividing panel. Twenty flies were placed in the half with the treated surface. Sugar and water was placed in both halves. The number of insects crossing from the treated area into the untreated area was assessed at 5 minute intervals for a total of 20 minutes.

For the testing against ants, cockroaches and bedbugs: A clear plastic container was used measuring approximately 34 cm long×21 cm wide×20 cm high. This was divided into two halves. The treated aluminium foil was placed in one half and the other half contained untreated foil. Food (sugar cube for black ants and bran pellet for cockroaches) and water (damp cotton wool) were placed in each half where appropriate. Twenty ants/cockroaches and ten bedbugs were placed in the centre of the arena. The number of insects in each half was assessed at 5 minute intervals for a total of 20 minutes.

Three to six replicates were performed for each treatment, for each species.

### Results

The results are shown in Table 3. If, at the indicated time, 10% or less of the insects were on the treated tile (or moving towards the treated tile, in the case of houseflies) it was assigned an A. If between 11% and 25% were on the treated tile (or moving towards the treated tile, in the case of houseflies) it was assigned a B. If greater than 25% were on the treated tile (or moving towards the treated tile, in the case of houseflies) it was assigned a C.

TABLE 3
Icaridin analogues

| | | COMPOUND | | | | |
|---|---|---|---|---|---|---|
| | TIME | Icaridin | | | | |
| SPECIES | (mins) | (38) | 39 | 40a | 42a | 41 |
| Black Ants | 5 | B | B | C | B | C |
| | 10 | B | B | C | B | C |
| | 15 | B | B | C | B | C |
| | 20 | A | B | C | A | C |
| Bedbugs | 5 | A | A | B | A | A |
| | 10 | A | B | A | A | B |
| | 15 | A | B | B | A | B |
| | 20 | A | A | B | A | B |
| German | 5 | B | B | C | B | B |
| Cockroaches | 10 | B | B | B | B | B |
| | 15 | A | B | B | B | B |
| | 20 | A | B | B | B | B |
| Houseflies | 5 | B | B | B | A | A |
| | 10 | C | C | B | A | A |
| | 15 | C | C | B | B | B |
| | 20 | C | C | B | B | B |

### Example 42

### Testing the Herbicidal Activity of XXXXX Analogues

A laboratory bioassay was conducted to screen ten compounds (Fluazifop (25); Ethyl fluazifop (62); five fluazifop analogues: 27, 28, 63, 64 and 65; mesofulfuron (6); and two mesosulfuron analogues: 8 and 61. for activity against Lolium perenne, barley, peas and Chinese cabbage. Compounds were diluted in DMSO. Controls were performed using both DMSO and water.

### Test Systems

Plants were obtained as seeds and were grown to the 2-4 true leaf stage. Plants were grown individually in seed trays. Each plant (in an approximately 3 cm diameter plug) was then detached from the tray for spraying. Environmental conditions were closely monitored and recorded and were within the optimal range of the target species.

### Test Treatments and Application

The compounds were screened at a range of six concentrations, eg. 0.05%, 0.01%, 0.005%, 0.001%, 0.0005% and 0.0001%. Treatments will be applied directly onto the plants, using a potter tower.

### Experimental Design

One plant of each type will be sprayed using a potter tower. The 4 different types of weeds will be placed in a 10 cm diameter area underneath the potter sprayer and sprayed simultaneously. The growth of the plants and any Phytotoxicity effects will then be assessed at specified growth intervals, according to EPPO guideline PP1/135. Five replicates will be performed for each treatment, for each species.

### Results

The results are shown in Tables 4 and 5. If a target weed species exhibited over 80% necrosis at a specified concentration at a particular time it was assigned an A, if it exhibited 50%-80% necrosis it was assigned a B and if it exhibited less than 50% necrosis, it was assigned a C.

TABLE 4
Fluazifop analogues

| Com- | | Day | Day | Day | Day | Day | Day | Day |
|---|---|---|---|---|---|---|---|---|
| pound | Dose | 0 | 1 | 3 | 7 | 10 | 14 | 21 |
| | | Sum of % necrosis in barley | | | | | | |
| Fluazifop | 0.000001 | C | C | C | C | C | C | C |
| Ethyl ester | 0.000005 | C | C | C | C | C | C | C |
| (62) | 0.00001 | C | C | C | C | C | C | B |
| | 0.00005 | C | C | C | C | C | C | B |
| | 0.0001 | C | C | C | C | C | C | A |
| | 0.0005 | C | C | C | C | C | C | A |
| Fluazifop | 0.000001 | C | C | C | C | C | C | C |
| (25) | 0.000005 | C | C | C | C | C | C | C |
| | 0.00001 | C | C | C | C | C | C | C |
| | 0.00005 | C | C | C | C | C | C | A |
| | 0.0001 | C | C | C | C | C | C | A |
| | 0.0005 | C | C | C | C | C | B | A |
| 27 | 0.000001 | C | | C | C | C | C | C |
| | 0.000005 | C | | C | C | C | C | C |
| | 0.00001 | C | | C | C | C | C | C |
| | 0.00005 | C | | C | C | C | C | C |
| | 0.0001 | C | | C | C | C | C | C |
| | 0.0005 | C | | C | C | C | C | C |
| 28 | 0.000001 | C | C | C | C | C | C | C |
| | 0.000005 | C | C | C | C | C | C | C |
| | 0.00001 | C | C | C | C | C | C | C |
| | 0.00005 | C | C | C | C | C | C | C |
| | 0.0001 | C | C | C | C | C | C | B |
| | 0.0005 | C | C | C | C | C | C | A |
| 63 | 0.000001 | C | C | C | C | C | C | C |
| | 0.000005 | C | C | C | C | C | C | C |
| | 0.00001 | C | C | C | C | C | C | C |
| | 0.00005 | C | C | C | C | C | B | B |
| | 0.0001 | C | C | C | C | C | C | C |
| | 0.0005 | C | C | C | C | C | C | C |
| 64 | 0.000001 | C | C | C | C | C | C | C |
| | 0.000005 | C | C | C | C | C | C | C |
| | 0.00001 | C | C | C | C | C | C | C |
| | 0.00005 | C | C | C | C | C | C | C |
| | 0.0001 | C | C | C | C | C | C | C |
| | 0.0005 | C | C | C | C | B | B | B |
| 65 | 0.000001 | C | C | C | C | C | C | |
| | 0.000005 | C | C | C | C | C | C | |
| | 0.00001 | C | C | C | C | C | C | |
| | 0.00005 | C | C | C | C | C | C | |
| | 0.0001 | C | C | C | C | C | C | |
| | 0.0005 | C | C | C | C | C | C | |
| | | Sum of % necrosis in Chinese cabbage | | | | | | |
| Fluazifop | 0.000001 | C | C | C | C | C | C | C |
| Ethyl ester | 0.000005 | C | C | C | C | A | A | A |
| (62) | 0.00001 | C | C | C | C | B | A | A |
| | 0.00005 | C | C | C | B | A | A | A |
| | 0.0001 | C | C | C | B | A | A | A |
| | 0.0005 | C | C | B | A | A | A | A |
| Fluazifop | 0.000001 | C | C | C | C | C | C | C |
| (25) | 0.000005 | C | C | C | C | B | B | B |
| | 0.00001 | C | C | B | B | A | A | A |
| | 0.00005 | C | B | B | A | A | A | A |
| | 0.0001 | C | B | A | A | A | A | A |
| | 0.0005 | C | C | B | A | A | A | A |
| 27 | 0.000001 | C | C | C | C | C | C | C |
| | 0.000005 | C | C | C | C | C | C | C |
| | 0.00001 | C | C | C | C | C | C | C |
| | 0.00005 | C | C | C | C | C | C | C |
| | 0.0001 | C | C | C | C | C | C | C |
| | 0.0005 | C | C | C | C | C | C | C |
| 28 | 0.000001 | C | C | C | A | A | A | A |
| | 0.000005 | C | C | C | A | A | A | A |
| | 0.00001 | C | C | C | A | A | A | A |
| | 0.00005 | C | C | C | A | A | A | A |
| | 0.0001 | C | C | C | A | A | A | A |
| | 0.0005 | C | C | B | A | A | A | A |
| 63 | 0.000001 | C | C | C | A | A | A | A |
| | 0.000005 | C | B | B | A | A | A | A |
| | 0.00001 | C | B | B | A | A | A | A |
| | 0.00005 | C | B | A | A | A | A | A |
| | 0.0001 | C | B | A | A | A | A | A |
| | 0.0005 | C | A | A | A | A | A | A |
| 64 | 0.000001 | C | C | A | A | A | A | |
| | 0.000005 | C | C | B | A | A | A | |
| | 0.00001 | C | C | A | A | A | A | |
| | 0.00005 | C | C | A | A | A | A | |
| | 0.0001 | C | C | B | A | A | A | |
| | 0.0005 | C | C | B | B | B | A | |
| 65 | 0.000001 | C | C | C | C | C | C | |
| | 0.000005 | C | C | C | C | C | C | |
| | 0.00001 | C | C | C | C | C | C | |
| | 0.00005 | C | C | C | C | B | A | |
| | 0.0001 | C | C | C | C | B | A | |
| | 0.0005 | C | C | C | C | B | A | |
| | | Sum of % necrosis in Rye grass | | | | | | |
| Fluazifop | 0.000001 | C | C | C | C | C | C | C |
| Ethyl ester | 0.000005 | C | C | C | C | C | C | C |
| (62) | 0.00001 | C | C | C | C | C | C | C |
| | 0.00005 | C | C | C | C | C | C | B |
| | 0.0001 | C | C | C | C | C | A | A |
| | 0.0005 | C | C | C | C | C | C | C |
| Fluazifop | 0.000001 | C | C | C | C | C | C | C |
| (25) | 0.000005 | C | C | C | C | C | C | C |
| | 0.00001 | C | C | C | C | C | C | B |
| | 0.00005 | C | C | C | C | C | C | B |
| | 0.0001 | C | C | C | C | C | C | B |
| | 0.0005 | C | C | C | C | C | A | A |
| 27 | 0.000001 | C | C | C | C | C | C | C |
| | 0.000005 | C | C | C | C | C | C | C |
| | 0.00001 | C | C | C | C | C | C | C |
| | 0.00005 | C | C | C | C | C | C | C |
| | 0.0001 | C | C | C | C | C | C | C |
| | 0.0005 | C | C | C | C | C | C | C |
| 28 | 0.000001 | C | C | C | C | C | C | C |
| | 0.000005 | C | C | C | C | C | C | C |
| | 0.00001 | C | C | C | C | C | C | C |
| | 0.00005 | C | C | C | C | C | C | C |
| | 0.0001 | C | C | C | C | C | C | C |
| | 0.0005 | C | C | C | C | C | B | A |
| 63 | 0.000001 | C | C | C | C | C | C | C |
| | 0.000005 | C | C | C | C | C | C | C |
| | 0.00001 | C | C | C | C | C | C | C |
| | 0.00005 | C | C | C | C | C | C | C |
| | 0.0001 | C | C | C | C | C | C | C |
| | 0.0005 | C | C | C | C | C | C | C |
| 64 | 0.000001 | C | C | C | C | C | C | C |
| | 0.000005 | C | C | C | C | C | C | C |
| | 0.00001 | C | C | C | C | C | C | C |
| | 0.00005 | C | C | C | C | C | C | C |
| | 0.0001 | C | C | C | C | C | C | C |
| | 0.0005 | C | C | C | C | C | C | C |
| 65 | 0.000001 | C | C | C | C | C | C | |
| | 0.000005 | C | C | C | C | C | C | |
| | 0.00001 | C | C | C | C | C | C | |
| | 0.00005 | C | C | C | C | C | C | |
| | 0.0001 | C | C | C | C | C | C | |
| | 0.0005 | C | C | C | C | C | C | |
| | | Sum of % necrosis in Peas | | | | | | |
| Fluazifop | 0.000001 | C | C | C | C | C | C | C |
| Ethyl ester | 0.000005 | C | C | C | C | C | B | B |
| (62) | 0.00001 | C | C | C | C | B | A | A |
| | 0.00005 | C | C | C | C | B | A | A |
| | 0.0001 | C | C | C | C | B | A | A |
| | 0.0005 | C | C | C | C | C | C | A |
| Fluazifop | 0.000001 | C | C | C | C | C | C | C |
| (25) | 0.000005 | C | C | C | C | C | C | C |
| | 0.00001 | C | C | C | C | B | B | A |
| | 0.00005 | C | C | C | C | C | C | A |
| | 0.0001 | C | C | C | C | C | C | A |
| | 0.0005 | C | C | C | C | C | C | A |
| 27 | 0.000001 | C | C | C | C | C | C | C |
| | 0.000005 | C | C | C | C | C | C | C |
| | 0.00001 | C | C | C | C | C | C | C |
| | 0.00005 | C | C | C | C | C | C | C |
| | 0.0001 | C | C | C | C | C | C | C |
| | 0.0005 | C | C | C | C | C | C | C |
| 28 | 0.000001 | C | C | C | C | C | C | B |
| | 0.000005 | C | C | C | B | B | B | A |
| | 0.00001 | C | C | C | C | C | A | A |
| | 0.00005 | C | C | C | C | C | A | A |
| | 0.0001 | C | C | C | C | C | A | A |
| | 0.0005 | C | C | C | B | B | A | A |
| 63 | 0.000001 | C | C | C | C | B | B | A |
| | 0.000005 | C | C | C | C | B | B | A |
| | 0.00001 | C | C | C | C | B | B | A |
| | 0.00005 | C | C | C | B | A | A | A |
| | 0.0001 | C | C | C | C | C | B | B |
| | 0.0005 | C | C | C | C | B | B | B |
| 64 | 0.000001 | C | C | C | C | C | C | B |
| | 0.000005 | C | C | C | C | C | C | C |
| | 0.00001 | C | C | C | C | C | B | A |
| | 0.00005 | C | C | C | C | C | C | C |
| | 0.0001 | C | C | C | C | C | B | A |
| | 0.0005 | C | C | C | C | C | C | A |
| 65 | 0.000001 | C | C | C | C | C | C | |
| | 0.000005 | C | C | C | C | C | C | |
| | 0.00001 | C | C | C | C | C | C | |
| | 0.00005 | C | C | C | C | C | C | |
| | 0.0001 | C | C | C | C | C | C | |
| | 0.0005 | C | C | C | C | C | C | |

TABLE 5
Mesosulfuron analogues

| | | | Day | Day | Day | Day | Day |
|---|---|---|---|---|---|---|---|
| Code | Dose | Day 0 | 1 | 3 | 7 | 10 | 14 |
| | | Sum of % necrosis in Barley | | | | | |
| Mesosulfuron | 0.000001 | C | C | C | C | C | C |
| (6) | 0.000005 | C | C | C | C | C | C |
| | 0.00001 | C | C | C | C | C | C |
| | 0.00005 | C | C | C | C | C | C |
| | 0.0001 | C | C | C | C | B | B |
| | 0.0005 | C | C | C | C | B | B |
| 8 | 0.000001 | C | C | C | C | C | C |
| | 0.000005 | C | C | C | C | C | C |
| | 0.00001 | C | C | C | C | C | C |
| | 0.00005 | C | C | C | C | C | C |
| | 0.0001 | C | C | C | C | C | C |
| | 0.0005 | C | C | C | C | C | C |
| 61 | 0.000001 | C | C | C | C | C | C |
| | 0.000005 | C | C | C | C | C | C |
| | 0.00001 | C | C | C | C | C | C |
| | 0.00005 | C | C | C | C | C | C |
| | 0.0001 | C | C | C | C | C | C |
| | 0.0005 | C | C | C | C | C | C |
| | | Sum of % necrosis in Chinese cabbage | | | | | |
| Mesosulfuron | 0.000001 | C | C | A | A | A | A |
| (6) | 0.000005 | C | C | A | A | A | A |
| | 0.00001 | C | C | A | A | A | A |
| | 0.00005 | C | C | A | A | A | A |
| | 0.0001 | C | C | A | A | A | A |
| | 0.0005 | C | C | A | A | A | A |
| 8 | 0.000001 | C | C | A | A | A | A |
| | 0.000005 | C | C | A | A | A | A |
| | 0.00001 | C | C | A | A | A | A |
| | 0.00005 | C | C | A | A | A | A |
| | 0.0001 | C | C | A | A | A | A |
| | 0.0005 | C | C | A | A | A | A |
| 61 | 0.000001 | C | C | C | C | C | C |
| | 0.000005 | C | C | C | C | C | B |
| | 0.00001 | C | C | C | C | B | B |
| | 0.00005 | C | C | C | C | C | B |
| | 0.0001 | C | C | C | C | B | B |
| | 0.0005 | C | C | C | C | B | B |
| | | Sum of % necrosis in Rye grass | | | | | |
| Mesosulfuron | 0.000001 | C | C | C | C | C | C |
| (6) | 0.000005 | C | C | C | C | C | C |
| | 0.00001 | C | C | C | C | C | C |
| | 0.00005 | C | C | C | C | B | B |
| | 0.0001 | C | C | C | C | B | A |
| | 0.0005 | C | C | C | C | B | A |
| 8 | 0.000001 | C | C | C | C | C | C |
| | 0.000005 | C | C | C | C | C | C |
| | 0.00001 | C | C | C | C | C | C |
| | 0.00005 | C | C | C | C | C | C |
| | 0.0001 | C | C | C | B | B | B |
| | 0.0005 | C | C | C | C | B | B |
| 61 | 0.000001 | C | C | C | C | C | C |
| | 0.000005 | C | C | C | C | C | C |
| | 0.00001 | C | C | C | C | C | C |
| | 0.00005 | C | C | C | C | C | C |
| | 0.0001 | C | C | C | C | C | C |
| | 0.0005 | C | C | C | C | C | C |
| | | Sum of % necrosis in Pea | | | | | |
| Mesosulfuron | 0.000001 | C | C | A | A | A | A |
| (6) | 0.000005 | C | C | B | A | A | A |
| | 0.00001 | C | C | A | A | A | A |
| | 0.00005 | C | C | B | A | A | A |
| | 0.0001 | C | C | A | A | A | A |
| | 0.0005 | C | C | C | A | A | A |
| 8 | 0.000001 | C | C | B | A | A | A |
| | 0.000005 | C | C | B | B | A | A |
| | 0.00001 | C | C | B | A | A | A |
| | 0.00005 | C | C | B | A | A | A |
| | 0.0001 | C | C | B | A | A | A |
| | 0.0005 | C | C | B | A | A | A |
| 61 | 0.000001 | C | C | C | C | C | C |
| | 0.000005 | C | C | C | C | C | C |
| | 0.00001 | C | C | C | C | C | B |
| | 0.00005 | C | C | C | C | B | B |
| | 0.0001 | C | C | C | C | C | C |
| | 0.0005 | C | C | C | B | B | A |

1. A compound of formula IIa: 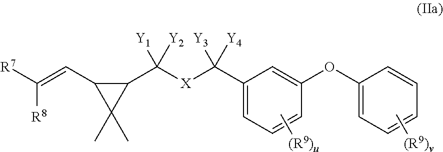
wherein X is NH, CH₂ or O;
wherein Y₁ is H and Y₂ is a group independently selected from W, OR⁵ and H and Y₃ and Y₄ together form a group independently selected from: ═O and ═NOR³; or
Y₃ is H and Y₄ is a group independently selected from W, OR⁵ and H and Y₁ and Y₂ together form a group independently selected from: ═O and ═NOR³; or
wherein 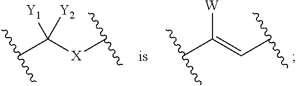
or wherein 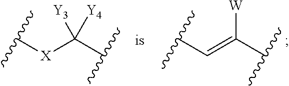
wherein W is a group independently selected from: H, CN, CO₂R⁵, CHO, CH═NOR³, CH(OR⁶)(OR⁶); CSNHR⁵, CH₂OR⁴, CONHR⁵;
or Y₂ and W, the atoms to which they are attached and the oxygen atom between the point of attachment of W and Y₂ together form a five membered ring in which two of the atoms in the ring are oxygen, and wherein the ring is optionally substituted with a group selected from: ═O or OR⁵;
R³ is independently a group selected from: H, C₁-C₄ alkyl, C₁-C₄ haloalkyl, phenyl, benzyl;
R⁴ is independently a group selected from: H and Ac;
R⁵ is independently at each occurrence a group selected from: H, C₁-C₄ alkyl, phenyl, benzyl;
R⁶ is independently at each occurrence a group selected from: C₁-C₄ alkyl, benzyl; or two R⁶ groups together with the atoms to which they are attached form a 5- or 6-membered ring;
R⁷ and R⁸ are a group independently selected from: halo and C₁-C₄ haloalkyl;
R⁹ is independently at each occurrence a group selected from: halo, C₁-C₄ alkyl, C₁-C₄-haloalkyl;
wherein each of the aforementioned alkyl, haloalkyl, phenyl and benzyl groups are optionally substituted, where chemically possible, by 1 to 3 substituents which are independently at each occurrence selected from: oxo, imino, oximo, halo, nitro, cyano, hydroxyl, amino, CO₂H, CO₂—(C₁-C₄alkyl), C(O)H, C₁-C₄-alkyl, C₁-C₄ haloalkyl, C₁-C₄ alkoxy, and C₁-C₄ haloalkoxy;
u is an integer selected from: 0, 1, 2, 3, 4; and
v is an integer selected from: 0, 1, 2, 3, 4, 5;
with the proviso that the compound is not a compound selected from:

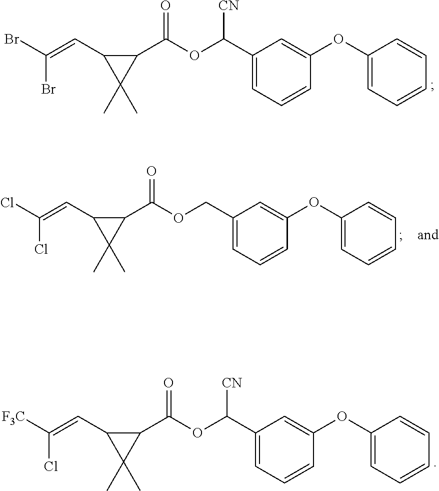 [SMILES: CC1(C)C(C=C(Br)Br)C1C(=O)OC(C#N)c1cccc(Oc2ccccc2)c1 ; CC1(C)C(C=C(Cl)Cl)C1C(=O)OCc1cccc(Oc2ccccc2)c1 ; CC1(C)C(/C=C(\Cl)C(F)(F)F)C1C(=O)OC(C#N)c1cccc(Oc2ccccc2)c1]

2. A compound according to claim 1, wherein the compound is a compound of formula IId:

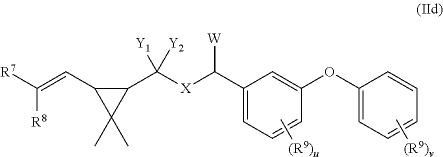

3. A compound according to claim 1, wherein X is CH₂ or NH.

4. A compound according to claim 1, wherein Y₁ and Y₂ together form ═O.

5. A compound according to claim 1, wherein the compound is selected from:

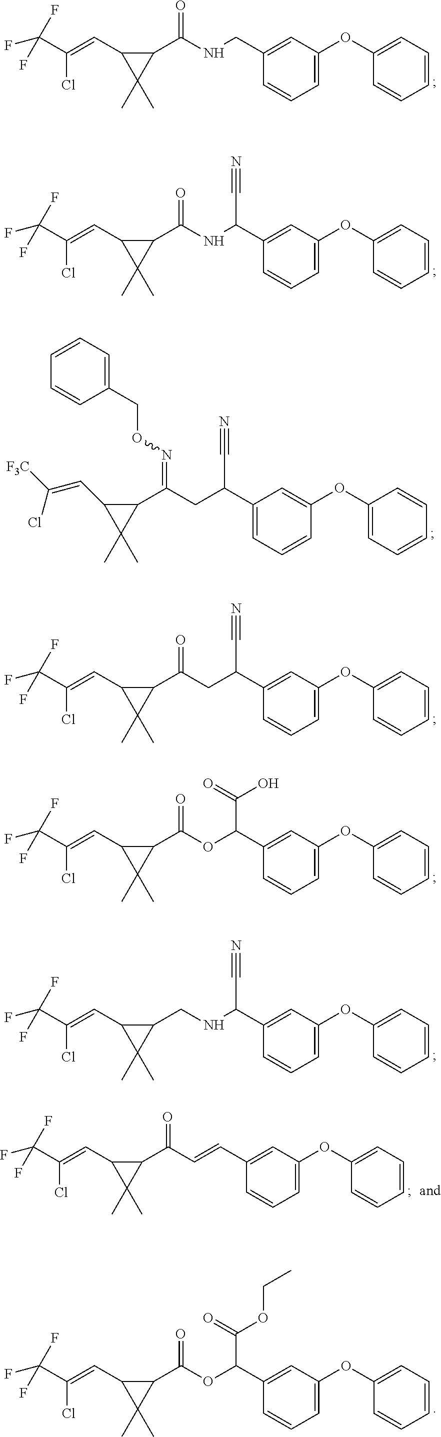

6.-12. (canceled)

13. A compound of formula XVI: 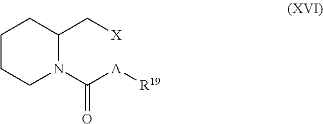
wherein X is a group independently selected from: CHO, CH═NOR³, CH(OR⁶)(OR⁶); CO₂R⁵;
A is a group selected from O, S and NH;
R³ is independently at each occurrence a group selected from: H, C₁-C₄ alkyl, C₁-C₄ haloalkyl, phenyl, benzyl;
R⁵ is independently at each occurrence a group selected from: H, C₁-C₄ alkyl, phenyl, benzyl;
R⁶ is independently at each occurrence a group selected from: C₁-C₄ alkyl, benzyl; or two R⁶ groups together with the atoms to which they are attached form a 5- or 6-membered ring; and
R¹⁹ is independently at each occurrence a group selected from: H, C₁-C₆ alkyl, C₁-C₄ haloalkyl, phenyl, benzyl; wherein each of the aforementioned alkyl, haloalkyl, phenyl and benzyl groups are optionally substituted, where chemically possible, by 1 to 3 substituents which are independently at each occurrence selected from: oxo, imino, oximo, halo, nitro, cyano, hydroxyl, amino, CO₂H, CO₂—(C₁-C₄alkyl), C(O)H, C₁-C₄-alkyl, C₁-C₄ haloalkyl, C₁-C₄ alkoxy, and C₁-C₄ haloalkoxy.

14. A compound according to claim 13, wherein the compound is a compound of formula XVII:

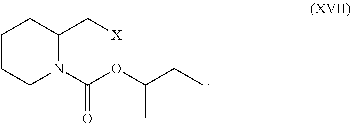

15. A compound according to claim 13, wherein the compound is a compound selected from:

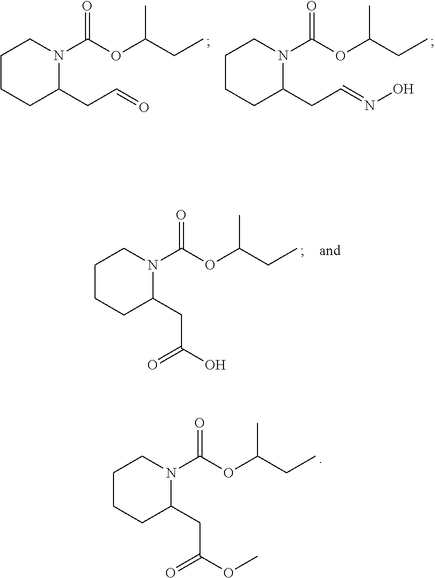

16. A compound of formula XVIII: 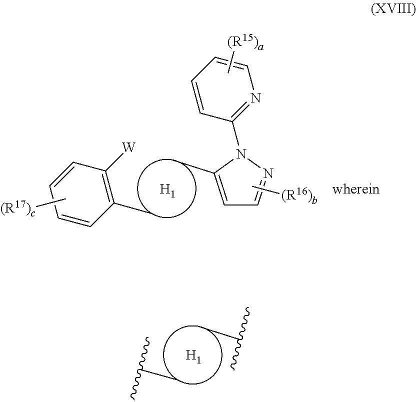
is a group selected from 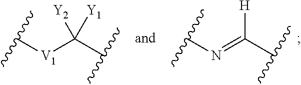
V₁ is a group independently selected from: O and NH;
Y₁ is H and Y₂ is independently at each occurrence a group selected from OR⁵ and H;
or Y₁ and Y₂ together form a group independently selected from: ═O and ═NOR³;
W is a group independently selected from: C(O)NR¹⁸R¹⁹, CHO, CO₂R⁵, CH═NOR³, CH(OR⁶)(OR⁶), heteroaryl, or CH₂OR⁴;
R³ is independently at each occurrence a group selected from: H, C₁-C₄ alkyl, C₁-C₄ haloalkyl, phenyl, benzyl;
R⁴ is independently a group selected from: H and Ac;
R⁵ is independently at each occurrence a group selected from: H, C₁-C₄ alkyl, phenyl, benzyl;
R⁶ is independently at each occurrence a group selected from C₁-C₄ alkyl, benzyl; or two R⁶ groups together with the atoms to which they are attached form a 5- or 6-membered ring;
R¹⁵, R¹⁶ and R¹⁷ are independently at each occurrence a group selected from: halo, C₁-C₄ alkyl, C₁-C₄-haloalkyl and cyano;
R¹⁸ and R¹⁹ are independently at each occurrence a group selected from: H, C₁-C₄ alkyl, phenyl, benzyl;
wherein each of the aforementioned alkyl, haloalkyl, phenyl and benzyl groups are optionally substituted, where chemically possible, by 1 to 3 substituents which are independently at each occurrence selected from: oxo, imino, oximo, halo, nitro, cyano, hydroxyl, amino, CO₂H, CO₂—(C₁-C₄alkyl), C(O)H, C₁-C₄-alkyl, C₁-C₄ haloalkyl, C₁-C₄ alkoxy, and C₁-C₄ haloalkoxy;
a is an integer independently selected from: 0, 1, 2, 3, 4;
b is an integer independently selected from: 0, 1, 2;
c is an integer independently selected from: 0, 1, 2, 3, 4,
with the proviso that if Y₁ and Y₂ together form ═O, W is not C(O)NHMe.

17. A compound according to claim 16, wherein the compound is a compound of formula (XX):

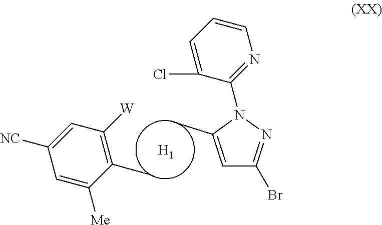

18. A compound according to claim 16, wherein

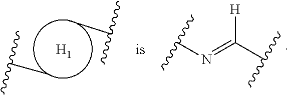

19. A compound according to claim 16, wherein W is CO₂R⁵.

20. A compound according to claim 16, wherein the compound is selected from: [chemical structure]
